(19) United States
(12) Patent Application Publication
(10) Pub. No.: US 2009/0036405 A1
(43) Pub. Date: Feb. 5, 2009

(54) METHOD FOR BLOCKING LIGATION OF THE RECEPTOR FOR ADVANCED GLYCATION END-PRODUCTS (RAGE)

(75) Inventor: Thomas P. Kennedy, Charlotte, NC (US)

Correspondence Address: ALSTON & BIRD LLP, BANK OF AMERICA PLAZA, 101 SOUTH TRYON STREET, SUITE 4000, CHARLOTTE, NC, 28280-4000, US

(73) Assignee: UNIVERSITY OF UTAH

(21) Appl. No.: 12/178,061

(22) Filed: Jul. 23, 2008

Related U.S. Application Data
(60) Provisional application No. 60/951,370, filed on Jul. 23, 2007.

(51) Int. Cl. A61K 31/727; A61P 25/28; A61P 9/00; A61P 17/18; A61P 9/10; A61P 19/02; A61P 27/02

(52) U.S. Cl. 514/56

(57) ABSTRACT

A method and medicament is provided for treating and inhibiting interaction of the receptor for advanced glycation end-products (RAGE) and its ligands using a natural or synthetic sulfated polysaccharide, preferably a 2-O desulfated heparin. The medicament preferably is administered intravenously, by aerosolization, intra-nasally, intra-articularly, intra-thecally, subcutaneously, topically or orally. The medicament is useful for treating a variety of conditions, including diabetes, inflammation, renal failure, aging, systemic amyloidosis, Alzheimer's disease, inflammatory arthritis, atherosclerosis, and colitis.

### CROSS-REFERENCE TO RELATED APPLICATIONS

The present application claims priority to U.S. Provisional Patent Application No. 60/951,370, filed Jul. 23, 2007, which is incorporated herein by reference in its entirety.

### FIELD OF THE INVENTION

The present invention relates to inhibition of ligation of the receptor for advanced glycation end-products (RAGE). More specifically, the invention relates to the use of a sulfated polysaccharide, such as a 2-O desulfated heparin, for inhibiting ligation of RAGE.

### BACKGROUND

The receptor for advanced glycation end-products (RAGE) is a multiligand receptor of the immunoglobulin superfamily. The receptor is comprised of immunoglobulin-like regions, including a distal “V” type domain where ligands bind, two “C” type domains, a short transmembrane domain, and a cytoplasmic tail required for signaling. RAGE is an important receptor developmentally as ligation by the DNA binding protein amphoterin (also known as HMGB1) is necessary for neural growth and development (Hori O, et al., J Biol Chem 1995; 270:25752-25761). However, RAGE also plays a part in many biological pathways not related to development.

One type of ligands known to bind to RAGE are advanced glycation end-products (AGEs), which are the chemical products of nonenzymatic attachment of sugars to proteins and lipids. AGEs accumulate in a plethora of biologic settings and have now been demonstrated to play important roles in the pathogenesis of a diverse array of diseases, including diabetes, inflammation, renal failure, aging, systemic amyloidosis, Alzheimer's dementia, inflammatory arthritis, atherosclerosis and colitis, to name but a few (Ramasamy R, et al., Glycobiology 2005; 15:16R-28R). In diabetes patients, AGEs form as a direct consequence of chronically elevated glucose, which proceeds through the polyol pathway to be reduced to sorbitol by the enzyme aldose reductase. Sorbitol is in turn converted to fructose, then fructose-3-phosphate, and then to 3-deoxyglucose, a reducing sugar whose aldehyde carbonyl can react in the Maillard reaction with the amino group of a target molecule such as an amino acid to form a Schiff base. The Schiff base can then undergo an intramolecular rearrangement to form Amadori products, which can further rearrange and condense to form fluorescent, yellow-brown products that represent AGEs (Wautier J-L, et al., Circ Res 2004; 95:233-238). A wide variety of chemical entities formed by these processes have been characterized, including amino acid cross links such as glycoxal-derived lysine dimer, hydroimidazolones such as methylglycoxal hydroimidazolone, and monolysyl adducts such as carboxymethyl-lysine (CML) and pyrraline.

The level of AGE product formation in diabetes is conveniently monitored by following the concentration of hemoglobin A1c, a naturally occurring minor human hemoglobin that is elevated in poorly controlled diabetic patients suffering chronic elevations of glucose, and thereby AGE formation. However, AGE products can also form in nondiabetic conditions as the result of oxidation reactions generated by oxidants such as hydrogen peroxide and hypochlorous acid released by activated phagocytes, or AGEs can be ingested from eating heavily cooked meats and other animal products (Huebschmann A G, et al., Diabetes Care 2006; 29:1420-1432). AGEs can even be formed in the lung as the consequence of cigarette smoke inhalation and its complicated oxidant chemistry (Carami C, et al., Proc Natl Acad Sci USA 1997; 94:13915-13920).

Rather than being specific for a single ligand, RAGE is a pattern recognition receptor that will bind a number of other ligands (Bierhaus A, et al., J Mol Med 2005; 83:876-886), including amyloid-β peptide (accumulating in Alzheimer's disease), amyloid A (accumulating in systemic amyloidosis), amphoterin (which is also released by necrotic macrophages and other cells in sepsis) and S100 calgranulins (a family of calcium-binding polypeptides that are released by phagocytes in sites of chronic inflammation). Once ligated and activated, RAGE mediates post-receptor signaling including activation of p21ras, ERK 1/2 (p44/p42) mitogen-activated protein (MAP) kinases, p38 and stress-activated/JNK kinases, rho GTPases, phosphoinositol-3 kinase, the JAK/STAT pathway, and activation of the transcription factors nuclear factor κB (NF-κB) and CREB (Yan S F, et al., Circ Res 2003; 93:1159-1169). These events, especially activation of NF-κB, lead to a profound inflammatory process, with up-regulated expression of a host of cytokines, including TNF-α, IL-1, IL-6, IL-8, GMCSF, adhesion molecules and inducible nitric oxide synthase. Furthermore, through the influence of a prominent NF-κB-responsive consensus sequence in its promoter, activation of RAGE also leads to even greater RAGE expression. In addition, RAGE can serve as an integrin-like endothelial attachment site mediating the efflux of phagocytes from the circulation into areas of inflammation. RAGE has been shown to interact with the leukocyte β2 integrins Mac-1 (CD11b/CD18) and p150,95 (CD11c/CD18) to facilitate phagocytic inflammatory cell recruitment (Chavakis T, et al., J Exp Med 2003; 198:1507-1515). The attraction of phagocytes to areas of inflammation is further augmented by interaction of the RAGE ligands S100 calgranulins and amphoterin (Orlova V V, et al., EMBO J 2007; 26:1129-1139). Thus, through local release of S100 and amphoterin (HMGB1), RAGE can amplify the inflammatory cascade with attraction of leukocytes to sites of inflammation. This leads to release of oxidants by the activated leukocytes, generation of more AGE products and sustained expression of additional pro-inflammatory mediators as additional RAGE is ligated and activated. Thus, RAGE can mediate a vicious cycle of sustained, smoldering inflammation in diseases where it is activated.

The importance of RAGE in disease has spurred vigorous attempts to inhibit activation of RAGE. One method has been to block formation of AGE products which bind and activate RAGE (Goldin A, et al., Circulation 2006; 114:597-605). The most promising agent for blocking formation of AGE products in human studies has been aminoguanidine. The hydrazine derivative aminoguanidine will react with 3-deoxyglucose, blocking formation of AGE products such as carboxymethyllysine. Aminoguanidine reduces AGE production and development of nephropathy and retinopathy in diabetic rats but produces glomerulonephritis in phase III human trials (Bolton W K, et al., Am J Nephrol 2004; 24:32-40). Other agents used experimentally to inhibit AGE formation include the vitamin derivatives pyridoxamine (a form of vitamin B6) and benfotiamine (a form of thiamine), the AGE cross link inhibitors N-2-acetaminodoethyl)hydrozinecarboximidamide hydrochloride (ALT-946), 4,5-dimethyl-3-phenyacylithiozolium chloride (ALT-711), and aldose reductase inhibitors such as epalrestat. Thus far, none has proven effective or safe in later stage human trials.

Experimentally, RAGE-mediated inflammation has been inhibited in animal models of diabetes or inflammation by daily injections of a recombinant form of the extracellular RAGE peptide comprised of the ligand binding domains but lacking transmembrane or cytoplasmic domains. This decoy receptor (so-called sRAGE for soluble RAGE) sponges up ligands such as amphoterin, AGEs, S100 proteins and leukocyte integrins such as Mac-1, competing against their interaction with native RAGE. In this fashion, sRAGE serves as an effective competitive inhibitor for RAGE-mediated inflammation. While sRAGE is effective at inhibiting RAGE in a number of animal models, though, it is a recombinant protein that is relatively expensive to manufacture compared to traditional organic compound based pharmaceutical drugs, and its safety in humans has not been tested. An effective inhibitor of RAGE-mediated inflammation would be expected to prove therapeutically useful in the treatment of a wide variety of pathogenic conditions. However, no such inhibitor is available that is also proven safe for use in humans.

Some research suggests that electrostatic charge interactions play an important role in ligand-RAGE binding, but the evidence is in many cases contradictory and confusing. In some studies, the interaction with RAGE by ligands such as amphoterin (Srikrishna G, et al., J Neurochem 2002; 80:998-1008), also known as high mobility box group protein-1 (HMGB-1), or S100 calgranulins (Srikrishna G, et al., J Immunol 2001; 166:4678-4688) is dependent on the presence of anionic N-glycans containing non-sialic acid carboxylate groups, and deglycosylation alone disrupts amphoterin and S100 binding to RAGE.

The COOH-terminal motif in amphoterin (amino acids 150-183) that is responsible for RAGE binding contains 13 cationic but only 4 anionic amino acids, making it a net cationic, positively charged sequence overall that might bind negatively charged sequences in receptor molecules (Huttunen H J, et al., Cancer Res 2002; 62:4804-4811). This would suggest that cationic positively charged amino acids on the external topography of RAGE ligands bind to anionic negatively charged carboxylate groups on the N-glycans of the receptor.

Other work directly conflicts with the hypothesis that positively charged groups on ligands interact with negatively charged N-glycan carboxylate groups on RAGE. The study of interactions between soluble sRAGE and Alzheimer's β-amyloid peptide by atomic force microscopy and molecular modeling suggests that sRAGE dimerizes to form a highly hydrophilic pocket containing an area dominated by positively charged cationic residues provided by 35 Arg, 30 Lys, 40 Lys and 75 Arg (Chaney M O, et al., Biochim Biophys Acta 2005; 1741:199-205). This model suggests that a negatively charged region on the N-terminal of Alzheimer's β-amyloid peptide binds to this cationic pocket in the RAGE dimmer. This positively charged pocket in the RAGE dimer is also postulated to serve an ionic trap for the docking of negatively charged carboxylate of ε-carboxymethylated lysyl (CML) residues of chemically formed AGEs. Thus, the prior art is unclear and conflicting as to the nature of charge-charge interactions between RAGE (positive or negative charge on RAGE) and its ligands (positive or negative charges on amphoterin, S100, Alzheimer's β-amyloid peptide, CML and other ligands).

### SUMMARY OF THE INVENTION

The present invention is directed to methods and medicaments for safe and effective inhibition of ligand interaction with RAGE. RAGE ligands, such as amphoterin, S100 calgranulins, AGEs, Alzheimer's β-amyloid peptide, and Mac-1 (CD11b/CD18), are thought to bind to RAGE through electrostatic interactions between cationic (positive) and anionic (negative) charges on the proteins or respective surface glycans. However, as pointed out above, the art is not clear concerning which charges are important. Moreover, there is ambiguity whether the respectively important cationic and anionic charges are present on the binding surface of RAGE or on its binding ligands.

Heparins are poly-anionic molecules. In general, removal of anionic charge from heparin by desulfation decreases the ability of the desulfated heparin to bind to a respective cationic protein compared to fully or over-sulfated heparins. As an example, progressive N- and O-desulfation of heparin eliminates the ability of the heparin derivative to inhibit virus attachment and infection to human cells (Walker S J, et al., J Virol 2002; 76:6909-6918).

The present invention shows that anticoagulant activity is not necessary for inhibition of RAGE-ligand interaction by a heparin or heparin derivative. The invention also describes several desulfated heparin derivatives with low anti-coagulant activity that still retain activity for inhibiting RAGE-ligand interactions. Various heparin analogs have been synthesized that have reduced anticoagulant activity, including over-O-sulfated heparin (i.e., heparin wherein all hydroxyl groups are substituted by sulfate groups); 2-O desulfated heparin; 2-O, 3-O desulfated heparin; N-desulfated/N-acetylated heparin; 6-O desulfated heparin; and carboxyl reduced heparin, among others. These are described and have been used in investigation of other anti-inflammatory effects of heparin that are unrelated to blockade of RAGE-ligand interactions. Other sulfated polysaccharides that will inhibit RAGE-ligand interaction include dextran sulfate and pentosan polysulfate.

Heparin, reduced anti-coagulant heparins and dextran sulfates can also be produced in a range of molecular polymeric sizes ranging from less than 1,000 to 15,000 Daltons and higher. A chemically synthesized pentasaccharide with full anticoagulant activity is also commercially available as fondaparinux sodium (commercially available as ARIXTRA®). A non-anticoagulant derivative can be produced by periodate oxidation followed by sodium borohyride reduction (Frank R D, et al., Thromb Haemostasis 2006; 96:802-806). This non-anticoagulant fondaparinux derivative, as well as other fondaparinux derivatives produced by 2-O desulfation, 6-O desulfation, carboxyl reduction, N-desulfation, or de novo synthesis with these chemical modifications, will also inhibit RAGE-ligand interactions and signaling.

Of reduced anti-coagulant heparins, the preferred drug substance for blocking RAGE-ligand interactions and signaling in humans and other mammalian species is 2-O desulfated heparin. As will be shown in the examples to follow, 2-O desulfated heparin not only has greatly reduced anticoagulant activity compared to heparin, therefore encompassing less risk from bleeding, but also has less risk of triggering the rare but potentially deadly side effect of heparin-induced thrombocytopenia.

In one embodiment, the present invention provides a method of inhibiting interaction or signaling between a ligand and RAGE. Preferably, the method comprises contacting RAGE with a sulfated polysaccharide. Most preferably, the sulfated polysaccharide comprises 2-O desulfated heparin. Even more preferably, the 2-O desulfated heparin is also 3-O desulfated. In particular embodiments, RAGE is contacted with the 2-O desulfated heparin in vivo. Thus, according to this aspect of the invention, the method can comprise administering the 2-O desulfated heparin to a patient, such as a mammal, preferably a human. Administration can be by any route effective to achieve in vivo contact of RAGE by the 2-O desulfated heparin.

According to another embodiment, the invention provides a method of treating a subject with a condition mediated by interaction or signaling between a ligand and RAGE. The method preferably comprises administering to the subject a sulfated polysaccharide, preferentially 2-O desulfated heparin. Even more preferentially, the 2-O desulfated heparin is also 3-O desulfated. According to this embodiment of the invention, the condition to be treated can encompass a wide variety of condition in light of the wide involvement of RAGE in multiple conditions. Non-limiting examples of conditions that can be treated according to the invention include diabetes, inflammation, renal failure, aging, systemic amyloidosis, Alzheimer's disease, inflammatory arthritis, atherosclerosis, colitis, periodontal diseases, psoriasis, atopic dermatitis, rosacea, multiple sclerosis, chronic obstructive pulmonary disease (COPD), cystic fibrosis, photoaging of the skin, age-related macular degeneration, and acute lung injury.

The ability to treat a wide variety of conditions according to the present invention is further characterized by the types of ligands that interact with or signal RAGE. For example, in certain embodiments, the present invention provides for treatment of conditions mediated by interaction or signaling of RAGE and a ligand selected from the group consisting of advanced glycation end products (AGEs), Alzheimer's β peptide, Amyloid proteins, S100 calgranulins, HMGB-1 (amphoterin), and Mac-1 integrin.

The ability to treat a wide variety of conditions according to the present invention is still further characterized by the types of enzymes or pathways that are activated or expressed by the interaction or signaling of RAGE and its ligands. For example, in certain embodiments, the present invention provides for treatment of conditions characterized by activation or expression of one or more enzymes or pathways selected from the group consisting of p21 ras, ERK 1/2 MAP kinases, JNK kinases, rho GTPases, phosphoinositol-3 kinase, JAK/STAT pathway, NF-κB, CREB, TNF-α, IL-1, IL-6, IL-8, GMCSF, iNOS, ICAM-1, E-selectin, VCAM-1, and VEGF.

### BRIEF DESCRIPTION OF THE DRAWINGS

Having thus described the invention in general terms, reference will now be made to the accompanying drawing, which is not necessarily drawn to scale, and wherein:

FIG. 1 shows a chemical formula of the pentasaccharide binding sequence of unfractionated heparin and the comparable sequence of 2-O, 3-O desulfated heparin (ODS heparin or ODSH);

FIG. 2 shows the differential molecular weight distribution plots determined by multiangle laser light scattering, in conjunction with high performance size exclusion chromatography, of the ODS heparin compared to the parent porcine intestinal heparin from which it was produced;

FIG. 3A and FIG. 3B shows disaccharide analysis of heparin and the ODS heparin of the present invention;

FIG. 4 shows a proposed reaction scheme for desulfating the 2-O position of α-L-iduronic acid in the pentasaccharide binding sequence of heparin;

FIG. 5 shows cross-reactivity of the 2-O desulfated heparin of this invention to heparin antibody, as determined by the serotonin release assay;

FIG. 6 shows cross-reactivity of the 2-O, 3-O desulfated heparin of this invention to heparin antibody, as determined by expression of platelet surface P-selectin (CD62) quantitated by flow cytometry;

FIG. 7 is a graph showing that increasing concentrations of 2-O desulfated heparin (which is also 3-O desulfated) suppressed HIT-mediated platelet activation, as shown by the release of platelet serotonin in response to adding 0.1 or 0.5 U/ml heparin to serum from a patient with HIT syndrome;

FIG. 8 is a graph showing mean results of experiments in which 2-O desulfated heparin (which is also 3-O desulfated) suppressed platelet activation, as shown by serotonin release induced by 0.1 U/ml heparin (UFH) in the presence of sera from four patients with HIT;

FIG. 9 shows a graph of the mean results of experiments in which 2-O desulfated heparin (which is also 3-O desulfated) suppressed platelet activation, as shown by serotonin release induced by 0.5 U/ml heparin (UFH) in the presence of sera from four patients with HIT;

FIG. 10 is a graph showing that 2-O desulfated heparin (which is also 3-O desulfated) suppressed platelet microparticle formation, when a HIT patient's serum is mixed with 0.1 U/ml or 0.5 U/ml heparin;

FIG. 11 is a graph showing mean results of experiments in which 2-O desulfated heparin (which is also 3-O desulfated) suppressed platelet microparticle formation, when sera from each of four patients with HIT is mixed with 0.1 U/ml heparin;

FIG. 12 is a graph showing mean results of experiments in which 2-O desulfated heparin (which is also 3-O desulfated) suppressed platelet microparticle formation, when sera from each of four patients with HIT is mixed with 0.5 U/ml heparin;

FIG. 13 is a graph showing that 2-O desulfated heparin (which is also 3-O desulfated) suppressed HIT-induced platelet activation, measured by platelet surface expression of P-selectin (CD62);

FIG. 14 is a graph showing mean results of experiments in which 2-O desulfated heparin (which is also 3-O desulfated) suppressed platelet surface expression of P-selectin (CD62), induced by HIT sera from each of four patients, with HIT in the presence of 0.1 U/ml unfractionated heparin;

FIG. 15 is a graph showing mean results of experiments in which 2-O desulfated heparin (which is also 3-O desulfated) suppressed platelet surface expression of P-selectin (CD62), induced by HIT sera from each of four patients, with HIT in the presence of 0.5 U/ml unfractionated heparin;

FIG. 16 is a graph showing blood concentrations of the preferred 2-O desulfated heparin, (which is also 3-O desulfated, termed ODSH), after the final injection into male beagle dogs in doses of 4 mg/kg every 6 hours (16 mg/kg/day), 12 mg/kg every 6 hours (48 mg/kg/day), and 24 mg/kg every 6 hours (96 mg/kg/day) for 10 days;

FIG. 17 shows inhibition of Mac-1 (CD11b/CD18) mediated attachment of U937 human monocytes to immobilized RAGE-Fc chimera by unfractionated heparin;

FIG. 18 shows inhibition of Mac-1 (CD11b/CD18) mediated attachment of U937 human monocytes to immobilized RAGE-Fc chimera by 2-O desulfated heparin, which is also 3-O desulfated (ODSH);

FIG. 19 shows inhibition of Mac-1 (CD11b/CD18) mediated attachment of U937 human monocytes to immobilized RAGE-Fc chimera by 6-O desulfated heparin;

FIG. 20 shows inhibition of Mac-1 (CD11b/CD18) mediated attachment of U937 human monocytes to immobilized RAGE-Fc chimera by N-desulfated heparin;

FIG. 21 shows inhibition of Mac-1 (CD11b/CD18) mediated attachment of U937 human monocytes to immobilized RAGE-Fc chimera by carboxyl-reduced heparin;

FIG. 22 shows inhibition of Mac-1 (CD11b/CD18) mediated attachment of U937 human monocytes to immobilized RAGE-Fc chimera by completely O-desulfated heparin;

FIG. 23 shows inhibition of Mac-1 (CD11b/CD18) mediated attachment of U937 human monocytes to immobilized RAGE-Fc chimera by low molecular weight heparin (average molecular weight of 5,000 daltons);

FIG. 24 shows inhibition of Mac-1 (CD11b/CD18) mediated attachment of U937 human monocytes to immobilized RAGE-Fc chimera by heparan sulfate;

FIG. 25 shows inhibition of Mac-1 (CD11b/CD18) mediated attachment of AMJ2C-11 alveolar macrophages to immobilized RAGE-Fc chimera by 2-O desulfated heparin, which is also 3-O desulfated (ODSH);

FIG. 26 shows inhibition of carboxymethyl-lysine bovine serum albumin (CML-BSA) binding to immobilized RAGE-Fc chimera by 2-O desulfated heparin, which is also 3-O desulfated (ODSH);

FIG. 27 shows inhibition of human S100b calgranulin binding to immobilized RAGE-Fc chimera by 2-O desulfated heparin, which is also 3-O desulfated (ODSH); and

FIG. 28 shows inhibition of human high mobility box group protein-1 (HMGB-1, or amphoterin) binding to immobilized RAGE-Fc chimera by 2-O desulfated heparin, which is also 3-O desulfated (ODSH).

### DETAILED DESCRIPTION OF THE INVENTION

The invention now will be described more fully hereinafter through reference to various embodiments. These embodiments are provided so that this disclosure will be thorough and complete, and will fully convey the scope of the invention to those skilled in the art. Indeed, the invention may be embodied in many different forms and should not be construed as limited to the embodiments set forth herein; rather, these embodiments are provided so that this disclosure will satisfy applicable legal requirements. As used in the specification, and in the appended claims, the singular forms “a”, “an”, “the”, include plural referents unless the context clearly dictates otherwise.

The present invention provides a safe and effective pathway for inhibiting ligation of a ligand to the receptor for advanced glycation end products (RAGE). Specifically, this is made possible through the use of sulfated polysaccharides, such as 2-O desulfated heparin. Contacting RAGE with a sulfated polysaccharide according to the invention effectively blocks the receptor and inhibits ligation with a variety of ligands, including those associated with many undesirable conditions, such as diabetes, inflammation, renal failure, aging, systemic amyloidosis, Alzheimer's disease, inflammatory arthritis, atherosclerosis, colitis, periodontal diseases, psoriasis, atopic dermatitis, rosacea, multiple sclerosis, chronic obstructive pulmonary disease (COPD), cystic fibrosis, photoaging of the skin, age-related macular degeneration, and acute lung injury.

As pointed out above, electrostatic charge interactions may play a role in ligand-RAGE binding. However, the contradictory and confusing research about the types of ionic charge interactions associated with RAGE ligation has hindered the identification of compounds useful as a RAGE ligation inhibiter with a wide variety of ligands.

Amphoterin has been shown to bind heparin (Salmivirta M, et al., Exp Cell Res 1992; 200:444-451; Rauvala H, et al., J Cell Biol 1988; 107:2293-2305; and Milev P, et al., J Biol Chem 1998; 273:6998-7005). Other RAGE ligands also bind to heparin, including S100 calgranulins (Robinson M J, et al, J Biol Chem 2002; 277:3658-3665) and the Alzheimer's amyloid-β peptide (Watson D J, et al., J Biol Chem 1997; 272:31617-31624; and McLaurin J, et al., Eur J Biochem 2000; 267:6353-6361). Heparin is also an adhesive ligand and inhibitor for the Mac-1 (CD11b/CD18) leukocyte integrin (Diamond M S, et al., J Cell Biol 1995; 130:1473-1482; and Peter K, et al., Circulation 1999; 100: 1533-1539). Dalteparin, a fully anticoagulant low molecular weight heparin, inhibits attachment of AGEs to RAGE in vitro and decreases AGE-stimulated signaling in endothelial cells leading to expression of mRNA for vascular endothelial growth factor and the integrin VCAM-1 (Myint K-M, et al., Diabetes 2006; 55:2510-2522). Thus, the ability of negatively charged heparins to reduce interaction of RAGE with the whole range of its ligands (including amphoterin, S100 calgranulins, Alzheimer's β-amyloid peptide, the leukocyte Mac-1 (CD11b/CD18) integrin, and AGEs) is consistent with disruption of charge-charge interactions between cationic sequences on the three-dimensional topography of RAGE ligands and negatively charged carboxylate groups of glycans found conjugated to the RAGE receptor surface.

Despite such evidence that heparins should be effective RAGE ligation inhibitors, there remains a failure in the art to provide an effective RAGE ligation inhibitor that is safe for use in humans for indications where anticoagulation is not desirable. For example, only unfractionated heparin and low-molecular weight heparins have been shown to block RAGE-ligand interactions. Unfractionated and low molecular weight heparins, though, retain full anticoagulant activity. It is therefore clear that the use of unfractionated and low molecular weight heparins as RAGE ligation inhibitors would present a serious risk of hemorrhage. A non-anticoagulant heparin derivative would be safer and therefore more clinically desirable to inhibit RAGE-ligand interactions and reduce the pathogenic effects of RAGE signaling.

No information exists on the effect of specific heparin modifications in relation to the activity of the modified heparin as an inhibitor of RAGE-ligand interactions and RAGE signaling. Furthermore, many modifications of heparin that decrease its anticoagulant activity also decrease the ability of the modified heparin to ionically bind specific biologic molecules and inhibit or stimulate their actions. However, there is no consistent theme to predict which heparin side groups are required to support a specific biologic interaction of heparin with a specific protein.

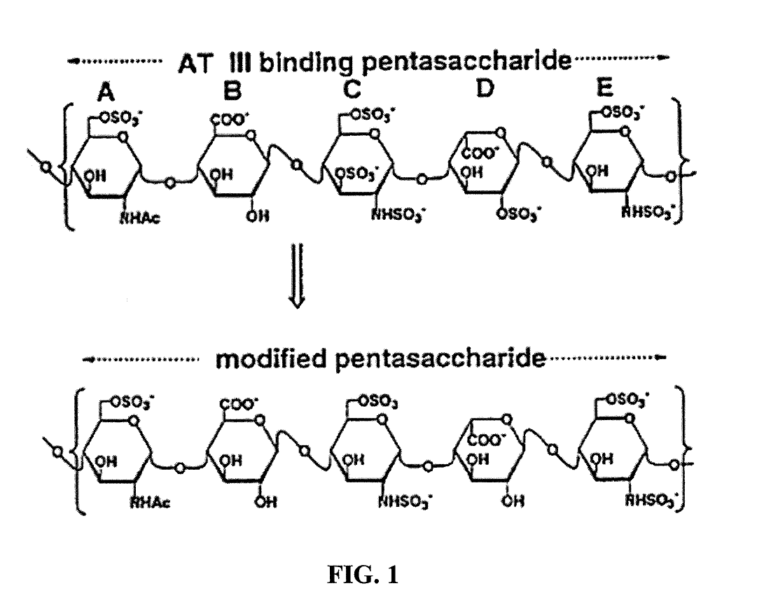

As examples, heparin competes with the attachment and internalization of a variety of viruses with human host cells. Selective removal of various polysaccharide side groups (FIG. 1) modifies this inhibitory activity, but which side groups are important for inhibition of viral attachment can vary from virus to virus. In the case of coxackievirus, unmodified and 2-O desulfated heparin inhibits coxackieviral cytopathic activity, but antiviral activity is markedly reduced by N- or 6-O desulfation (Zautner A E, et al., J Virol 2006; 80:6629-6636). In the case of herpes simplex virus (HSV), whereas N-desulfation or carboxyl reduction reduces heparin's antiviral activity for both HSV-1 and HSV-2, removal of 2-O, 3-O or 6-O sulfates significantly reduces the antiviral activity for HSV-1 but has little effect on the antiviral activity against HSV-2 (Herold B C, et al., J Virol 1996; 70:3461-3469). In the case of pseudorabies virus, different virus mutants exhibit different patterns of susceptibility to inhibition by selectively N-, 2-O, or 6-O desulfated heparins in a virus attachment/infectivity assay (Trybala E, et al., J Biol Chem 1998; 273:5047-5052).

Heparin also binds to the family of fibroblast growth factors (FGFs) and other growth factors, enhancing their activity in promoting wound healing by stimulating ERK 1/2 phosphorylation and proliferation in a variety of cell types. FGF family members differ greatly in the heparin sulfate groups required for inter-active support proliferative activity. FGF2 needs 2-O sulfate but not 6-O sulfate; FGF10 needs 6-O sulfate but not 2-O sulfate; FGF18 and hepatocyte growth factor have affinity for both 2-O sulfate and 6-O sulfate but prefer 2-O sulfate; and FGF4 and FGF7 require both 2-O and 6-O sulfate (Ashikari-Hada S, et al., J Biol Chem 2004; 279:12346-12354).

Heparin has potent anti-inflammatory activities dependent in part on its ability to block cationic leukocyte proteases, and in part on its ability to inhibit P- and L-selectins, integrins that determine initial attachment of platelets and leukocytes to the vascular endothelial cell surface and mediate leukocyte rolling. In the case of human leukocyte elastase (HLE), N-sulfate is required for inhibition, with N-desulfated heparin showing little functional HLE inhibitory activity (Fryer A, et al., J Pharmacol Exp Ther 1997; 282:208-219). In contrast, heparin inhibition of P- and L-selectins requires 6-O sulfation, with 6-O desulfated heparin losing much of its ability to inhibit leukocyte migration into areas of inflammation (Wang L, et al., J Clin Invest 2002; 110: 127-136).

Size also matters in the ability of a heparin to affect protein-protein interactions important for biologic function, but not in a predictable manner. In the case of FGF8b, heparins of greater than 14 monosaccharides are required for optimal activity, but in cells stimulated with FGF1 or FGF2, shorter heparins of only 6 to 8 monosaccharides will support proliferation (Loo B-M, et al., J Biol Chem 2002; 277:32616-32623). Unfractionated heparin is an efficient inhibitor of P- and L-selectins at concentrations usually present in the blood during therapeutic anti-coagulation, but currently available low molecular weight heparins do not effectively block P- and L-selectins at concentrations that produce similar levels of anti-coagulation (Koenig A, et al., J Clin Invest 1998; 101:877-889). In the case of RAGE, whereas larger unfractionated heparin has been reported to be less effective, the low molecular weight heparin dalteparin is a potent inhibitor of AGE-RAGE interaction.

These examples illustrate that side group modifications and size modifications greatly influence heparin's ability to bind to various proteins and enhance or inhibit that protein's actions. However, removal of a specific sulfate or reduction of its carboxyl does not affect the activity of heparin in a predictable manner. Each interaction of heparin with a specific protein is unique.

In relation to inhibiting RAGE-ligand interactions, there is no precedent for determining whether the removal of a specific sulfate or carboxyl to reduce anti-coagulant activity will also adversely affect the ability of that desulfated or carboxyl reduced heparin to inhibit RAGE-ligand activity in disease. However, in light of the art around ionic interactions in RAGE ligand binding, it would be predicted that any desulfation would serve to greatly reduce the activity of heparin to inhibit the charge-charge electrostatic interactions that appear important in RAGE-ligand binding. The present invention surprisingly shows that specific non-anticoagulant heparins are effective for inhibiting ligation of RAGE with the whole range of its ligands. This is illustrated below in the Examples showing empirical experimentation with a variety of desulfated and carboxyl reduced heparins to determine their ability to inhibit RAGE-ligand activity, using Mac-1 (CD11b/CD18)-mediated attachment of U937 human monocytes to immobilized RAGE as a paradigm RAGE-ligand interaction. Further examples show reduction of binding of in relation to other ligands, such as CML-BSA, HMGB-1, and S100b calgranulin. Those examples show wide and surprising differences in the requirement of various heparin side groups and heparin sizes for inhibition of ligand-RAGE interaction.

In addition to blocking RAGE-ligand interactions at the cellular membrane levels, 2-O desulfated heparin will also bind sRAGE, prolonging its half-life. This will serve to sustain the presence sRAGE longer in the extracellular matrix so that it can act as an effective decoy for ligands in opposition to cellular membrane RAGE, and act as a buffer to halt detrimental ligand-RAGE interactions.

As previously noted, only the fully anticoagulant low molecular weight heparin dalteparin has previously been found to be an effective inhibitor of RAGE-ligand interactions. Dalteparin sodium (known commercially as FRAGMIN®) is an injectable low molecular weight heparin produced through controlled nitrous acid depolymerization of unfractionated porcine intestinal heparin. The average molecular weight is 5,000 daltons, with only 14-26% of its polysaccharides weighing greater than 8,000 daltons (as described in the Physician's Desk Reference, 61^{st} edition. Medical Economics Co, Inc., Montvale, N.J. 2007, p 1097-1101). Dalteparin is fully anticoagulant against Factor Xa in the coagulation cascade with an anti-Xa activity of 156 U/mg. The major adverse reaction to dalteparin when given to humans is excessive hemorrhage as the consequence of its full anticoagulant activity.

With less than 10 U anti-Xa activity/mg, 2-O desulfated heparin, which is also 3-O desulfated, presents much less risk of adverse bleeding than dalteparin or other fully anticoagulant unfractionated or low molecular weight heparins. Because anticoagulation is not a desired therapeutic objective in treating or preventing RAGE-ligand interactions, 2-O desulfated heparin provides superior therapeutic safety as an inhibitor of RAGE-ligand interactions, compared to dalteparin or other fully anticoagulant heparins.

That a low anticoagulant heparin such as 2-O desulfated heparin can inhibit RAGE-ligand interactions and signaling is surprising since only low molecular weight heparin, such as dalteparin, has previously been shown to be effective for inhibiting RAGE-ligand interactions and signaling. This is particularly important since the anticoagulant activity of heparin is primarily based upon its ability to bind the blood serine proteinase inhibitor protein anti-thrombin III (ATIII), greatly increasing the potency of ATIII as an inhibitor of thrombin and coagulation factor Xa. While ATIII binding activity is primarily responsible for the anticoagulant activity of unfractionated and low molecular weight heparin, ATIII binding is also important for other nonanticoagulant functions of heparin. As an example, heparin stimulates the binding of fibroblast growth factors (FGF) with their respective receptor kinases (FGFR) to stimulate cell proliferation important in wound repair. Only that fraction of heparins and liver-derived heparan sulfate which bind ATIII facilitates formation of an active FGF-FGFR complex (McKeehan M L, et al., J Biol Chem 1999; 274:21511-21514). The prior art has failed to show that ATIII binding by dalteparin or heparin is unnecessary for inhibiting RAGE-ligand interactions. Thus, it is a surprise that a heparin compound having reduced ATIII binding activity (and therefore low anticoagulant activity), such as 2-O desulfated heparin, is an effective inhibitor of RAGE-ligand interaction and signaling.

While reduced in its degree of sulfation compared to fully anticoagulant heparins, it is even more surprising according to the invention that 2-O desulfated heparin, which is also 3-O desulfated, is an even more potent inhibitor of RAGE-ligand interaction than is fully anticoagulant low-molecular weight heparin. It is also a surprise that 2-O desulfated heparin is also a more potent inhibitor of RAGE-ligand interactions and signaling than other modifications of heparin which reduce anticoagulant activity by desulfation or carboxylate reduction, including 6-O desulfated heparin, N-desulfated heparin, carboxyl-reduced heparin, or fully desulfated heparin. Furthermore, it is a surprise that 2-O desulfated heparin, which is also 3-O desulfated, and is reduced in degree of sulfation and anionic charge compared to native unfractionated heparin, is more potent as an inhibitor of RAGE-ligand interactions and signaling than heparan sulfate, a naturally occurring low-anticoagulant sulfated polysaccharide that is also an inhibitor of RAGE-ligand interactions and signaling. These surprising results are more fully described in the Examples below.

It is further undesirable to use fully anticoagulant heparins as RAGE ligation inhibitors because of the associated heparin-induced thrombocytopenia (HIT) type 2. HIT is a dreaded complication of heparin therapy in which the binding of heparin to platelet factor 4 (PF4) elicits a conformational change in PF4 so that a previously quiescent antibody present in a minority of patients can bind to the heparin-PF4 complex. When the HIT antibody binds to heparin-PF4 complexes on the surface of platelets, the platelet becomes activated to aggregate (Levy J H, et al., Hematol Oncol Clinics North America 2007; 21:65-88). All currently available anticoagulant heparins (including dalteparin and unfractionated heparin), as well as nonanticoagulant heparins, can produce type 2 HIT in a susceptible individual. The only known exception is 2-O desulfated heparin. The present invention is thus even more advantageous in that 2-O desulfated heparin can be used as an inhibitor of RAGE-ligand interactions without the fear of activating HIT in a susceptible individual. This property also renders 2-O desulfated heparin a safer therapeutic approach to inhibiting RAGE-ligand interactions and signaling in patients.

While 2-O desulfated heparin is particularly preferred according to the invention, other types of sulfated polysaccharides also can be used, including heparin, various forms of reduced anticoagulant heparin (N-desulfated; 2-O, 3-O or 6-O desulfated; N-desulfated and reacetylated; O-decarboxylated; and over O-sulfated heparin), heparin sulfate, heparan sulfate, pentosan polysulfate, dextran sulfate and the pentasaccharide fondaparinux. General description of these compounds can be found, for example, in Wang L, et al., J Clin Invest 2002; 110: 127-136. While the invention may be described herein in relation to 2-O desulfated heparin or 2-O, 3-O desulfated heparin, such description is not intended to necessarily limit the scope of the invention but is rather provided as illustration of one embodiment of the invention.

The present invention is particularly beneficial in that it provides methods and medicaments for inhibiting interaction of RAGE with its ligands, including HMGB-1 (amphoterin), S100 calgranulins, AGEs, Alzheimer's β-amyloid peptides, other amyloid proteins, and the Mac-1 (CD11b/CD18) leukocyte integrin, among others, blocking the ability of these ligands to activate the RAGE receptor in a variety of tissues, organ systems and disease states.

In a particularly preferred embodiment of the present invention, the RAGE ligation inhibitor is 2-O desulfated heparin that is also 3-O desulfated. 2-O desulfated heparin that is also 3-O desulfated is a heparin analog with reduced anionic charge from its selective desulfation. Surprisingly, the present invention shows that 2-O desulfated heparin is a more potent inhibitor of RAGE-ligand interactions than even heparin or low molecular weight heparins. This is unexpected in light of the lower anionic charge of 2-O desulfated heparin, which would be predicted to reduce its RAGE-ligand inhibitor activity.

2-O desulfated heparin is further beneficial because of activity that is unrelated to inhibition of RAGE-ligand interactions and signaling. For example, 2-O desulfated heparin is anti-inflammatory by other mechanisms such as inhibiting the destructive effects of human leukocyte elastase (HLE) on a lung when instilled into the tracheal. Also unrelated to inhibition of RAGE-ligand interactions and signaling, the 2-O desulfated heparin inhibits binding of inflammatory cells, such as polymorphonuclear leukocytes and monocytes, to endothelium and platelets by blocking L- and P-selectins. The 2-O desulfated heparin of the present invention has the advantage of inhibiting RAGE-ligand interactions while having reduced anticoagulant activity, thereby eliminating the side effect of excessive anticoagulation that would result from equivalent doses of unmodified heparin. Moreover, as previously pointed out, other heparins and sulfated polysaccharides react with heparin antibodies often present in mammalian organisms to form glycosaminoglycan-platelet factor 4 (PF4)-HIT reactive antibody complexes capable of inducing platelet activation and the HIT type 2 thrombotic syndrome. The 2-O desulfated heparin of the present invention also has the advantage of inhibiting RAGE-ligand interactions without the side effect of HIT-2 thrombotic syndrome.

The 2-O desulfated heparin used in the present invention can have varying degrees of desulfation. Moreover, when the 2-O desulfated heparin is also 3-O desulfated, the degree of desulfation at the 2-O and 3-O positions can also vary. In preferred embodiments, the O-desulfated heparin is at least about 10%, at least about 25%, at least about 50%, at least about 75%, at least about 80%, at least about 90%, at least about 95%, at least about 97%, or at least about 98%, independently, at each of the 2-O position and the 3-O position. In specific embodiments, the O-desulfated heparin is 100% desulfated at one or both of the 2-O and the 3-O position. The extent of O-desulfation need not be the same at each O-position. For example, the heparin could be predominately (or completely) desulfated at the 2-O position and have a lesser degree of desulfation at the 3-O position. In one embodiment, the heparin is at least about 90% desulfated at both the 2-O and 3-O positions. The extent of O-desulfation or N-desulfation can be determined by known methods, such as disaccharide analysis.

One method of preparing O-desulfated heparin is provided in U.S. Pat. No. 5,990,097, which is herein by reference in its entirety. In the method disclosed therein, a 5% aqueous solution of porcine intestinal mucosal sodium heparin is made by adding 500 gm heparin to 10 L deionized water. Sodium borohydride is added to a 1% final concentration and the mixture is incubated. Sodium hydroxide is then added to a 0.4 M final concentration (pH at least 13) and the mixture is frozen and lyophilized to dryness. Excess sodium borohydride and sodium hydroxide can be removed by ultrafiltration. The final product is pH adjusted, cold ethanol precipitated, and dried. The O-desulfated heparin produced by this procedure is a fine crystalline slightly off-white powder with less than 10 USP units/mg anti-coagulant activity and less than 10 U/mg anti-Xa anti-coagulant activity.

The synthesis of O-desulfated heparin as described above can also include various modifications. For example, the starting heparin can be place in, for example, water, or other solvent, as long as the solution is not highly alkaline. A typical concentration of heparin solution can be from 1 to 10 percent by weight heparin. The heparin used in the reaction can be obtained from numerous sources, known in the art, such as porcine intestine or beef lung. The heparin can also be modified heparin, such as the analogs and derivatives described herein.

The heparin can be reduced by incubating it with a reducing agent, such a sodium borohydride, catalytic hydrogen, or lithium aluminum hydride. A preferred reduction of heparin is performed by incubating the heparin with sodium borohydride. Generally, about 10 grams of NaBH₄ can be used per liter of solution, but this amount can be varied as long as reduction of the heparin occurs. Additionally, other known reducing agents can be utilized but are not necessary for producing a treatment effective O-desulfated heparin. The incubation can be achieved over a wide range of temperatures, taking care that the temperature is not so high that the heparin caramelizes. Exemplary temperature ranges are about 15-30° C. or about 20-25° C. The length of the incubation can also vary over a wide range, as long as it is sufficient for reduction to occur. For example, several hours to overnight (i.e., about 4 to 12 hours) can be sufficient. However, the time can be extended to over several days, for example, exceeding about 60 hours.

Additionally, the method of synthesis can be adapted by raising the pH of the reduced solution to 13 or greater by adding a base capable of raising the pH to 13 or greater to the reduced heparin solution. The pH can be raised by adding any of a number of agents including hydroxides, such as sodium, potassium or barium hydroxide. A preferred agent is sodium hydroxide (NaOH). Even once a pH of 13 or greater has been achieved, it can be beneficial to further increase the concentration of the base. For example, it is preferable to add NaOH to a concentration of about 0.25 M to about 0.5 M NaOH. This alkaline solution is then dried, lyophilized or vacuum distilled.

In specific embodiments, the alkaline solution can comprise heparin and base in defined ratios. For example, when NaOH is used as the base, the ratio of NaOH to heparin (NaOH:heparin, in grams) can be about 0.5:1, preferably about 0.6:0.95, more preferably about 0.7:0.9. Of course, greater concentrations of base can be added, as necessary, to ensure the pH of the solution is at least 13.

Additional examples of the preparation of 2-O desulfated nonanticoagulant heparin, which is also 3-O desulfated, may be found in, for example, U.S. Pat. No. 5,668,188; U.S. Pat. No. 5,912,237; and U.S. Pat. No. 6,489,311, all of which are incorporated herein by reference. Yet further examples of the preparation of various forms of reduced anticoagulant heparin are found in Wang L, et al., J Clin Invest 2002; 110:127-136, which is incorporated herein by reference. Heparin, prepared from either porcine intestine or bovine lung, is available as a U.S.P. pharmaceutical from a number of manufacturers, including Scientific Protein Labs, Wanaukee, Wis. A number of methods, including alkaline depolymerization, periodate oxidation, nitrous acid depolymerization and treatment with bacterial heparinases are well known to those skilled in the art for reducing the average molecular weight size of unfractionated heparin to heparin fragments ranging from 6,000 down to as low as 1,000 Daltons. Dextran sulfate, having a variety of molecular weights and degrees of sulfation ranging in size from 5,000 to over 1,000,000 Daltons and suitable for use as an inhibitor the interaction of RAGE with its ligands, is available from a number of manufacturers, including Polydex Pharmaceuticals, Ltd, Nassau, Bahamas. Pentosan polysulfate can be obtained from IVAX Pharmaceuticals, Miami, Fla.

Small molecular weight synthetic inhibitors of RAGE-ligand interaction and signaling can also be produced starting with the synthetic pentasaccharide fondaparinux sodium. Fondaparinux can be synthesized by methods readily available in the literature (Choay J, et al., Biochem Biophys Res Comm 1983; 116:492-499; and Petitous M, et al., Carbohydrate Res 1986; 147:221-326). The resulting fully anticoagulant pentasaccharide can then be derivatized to a pentasaccharide with low anticoagulant activity but preserved inhibitory activity against RAGE-ligand interactions by N-desulfation, carboxyl reduction, 6-O desulfation or 2-O desulfation using chemical methods widely known in the art or described in detail above for 2-O desuflation. Alternately, the N-desulfated, 6-O desulfated, carboxyl reduced or 2-O, 3-O desulfated derivatives of fondaparinux can be synthesized de novo using obvious modifications of methods presented in detail.

Another method of manufacturing an effective inhibitor of RAGE-ligand interactions and signaling is based upon biosynthetic production of heparins starting with the biosynthetic K5 capsular polysaccharide purified from Escherichia coli, and modified to produce a heparin-like polysaccharide through progressive N-sulfation, N-deacetylation, C5 epimerization, per-O-sulfation, selective O-desulfation and 6-O-resulfation, producing a synthetic heparin-like polysaccharide (Lindahl U, et al., J Med Chem 2005; 48:349-352; and Rusnati M, et al., Current Pharmaceutical Design 2005; 11:2489-2499). This fully anticoagulant biosynthetic heparin can be subsequently modified by 2-O desulfation methods outlined above, which also produce 3-O desulfation, to produce an inhibitor of RAGE-ligand interactions and signaling with low anticoagulant activity and risk of bleeding. Alternately, the 6-O sulfation step can be eliminated, or the biosynthetic heparin can be treated by methods to effect N-desulfation or carboxyl reduction, well-known in the art, to also effect production of low anticoagulant inhibitors of RAGE-ligand interaction and signaling.

Under certain conditions, low molecular weight inhibitors of RAGE-ligand interactions and signaling might prove useful because of their favorable pharmacokinetics, allowing for rapid absorption, sustained blood levels and almost exclusive renal clearance following subcutaneous injection. Renal clearance might also prove useful in targeting RAGE-ligand interactions in the kidney. Low molecular weight versions of the sulfated polysaccharides discussed above can be easily produced using beta-elimination, alkaline depolymerization, periodate oxidant, nitrous acid depolymerization or treatment with bacterial heparinases. All three methods are well-known in the art, with an abundant literature.

Heparin is a heterogeneous mixture of variably sulfated polysaccharide chains composed of repeating units of D-glucosamine and either L-iduronic acid or D-glucuronic acids. The average molecular weight of heparin typically ranges from about 6,000 Da to about 30,000 Da, although certain fractions of unaltered heparin can have a molecular weight as low as about 1,000 Da. According to certain embodiments of the invention, heparin can have a molecular weight in the range of about 1,000 Da to about 30,000 Da, about 3,000 Da to about 25,000 Da, about 8,000 Da to about 20,000 Da, or about 10,000 Da to about 18,000 Da. Unless otherwise noted, molecular weight is expressed herein as weight average molecular weight (M_{W}), which is defined by formula (I) below

$${M}_{W} = \frac{\sum {n}_{i} {M}_{i}^{2}}{\sum {n}_{i} {M}_{i}} , \qquad ( I )$$

wherein nᵢ is the number of polymer molecules (or the number of moles of those molecules) having molecular weight Mᵢ.

The O-desulfated heparin used according to the invention can also have a reduced molecular weight so long as it retains the useful activity as described herein. Low molecular weight heparins can be made enzymatically by utilizing heparinase enzymes to cleave heparin into smaller fragments, or by depolymerization using nitrous acid. Such reduced molecular weight O-desulfated heparin can typically have a molecular weight in the range of about 100 Da to about 8,000 Da. In specific embodiments, the heparin used in the invention has a molecular weight in the range of about 100 Da to about 30,000 Da, about 100 Da to about 20,000 Da, about 100 Da to about 10,000 Da, about 100 to about 8,000 Da, about 1,000 Da to about 8,000 Da, about 2,000 Da to about 8,000 Da, or about 2,500 Da to about 8,000 Da.

One embodiment of a 2-O desulfated heparin that is also largely 3-O desulfated is illustrated in FIG. 1. In a specific embodiment, such 2-O, 3-O desulfated heparin can be prepared from unfractionated porcine heparin with an average molecular weight of 11,500 Da. This can then be reduced with sodium borohydride prior to lyophilization, the resulting product has an average molecular weight of about 10,500 Da.

In certain embodiments, the present invention provides a pharmaceutical composition comprising a sulfated polysaccharide useful for inhibiting interaction or signaling of ligands and RAGE. Preferably, the composition comprises 2-O desulfated heparin, more preferably 2-O, 3-O desulfated heparin.

As previously pointed out, the present invention is particularly surprising in that it shows that non-anticoagulant sulfated polysaccharides having reduced ability to inhibit blood coagulation compared to unfractionated and low molecular weight heparins, especially 2-O desulfated heparin, which is also 3-O desulfated, can be used to block interaction of RAGE with its ligands. This is particularly beneficial as the invention thus provides methods for treating a number of conditions affecting a wide variety of subjects, especially human subjects.

The ability of the invention to provide for treatment of a large number of conditions arises from the broad interaction of RAGE with a large number of ligands. Specifically, RAGE interacts with ligands involved in a wide range of diseases and undesirable conditions for which treatment is sought. Accordingly, as the present invention provides compounds that bind to RAGE and thus generally prevent RAGE from interacting with other ligands, the present invention is useful for treating the many conditions associated with these blocked ligands.

In particular embodiments, the methods of the present invention are useful in inhibiting interaction or signaling between RAGE and one or more ligands including, but not limited to, advanced glycation end-products (AGEs), amphoterin (also known as high-mobility group-box protein 1, or HMGB-1), S100 calgranulins, the Alzheimer's β-amyloid peptide, and the Mac-1 (CD11b/CD18) integrin of phagocytic cells, among others.

Interaction of AGEs with RAGE has been shown to modulate activities in many cell types. For example, in endothelial cells, AGE-RAGE interaction modulates the expression of adhesion molecules and the expression of proinflammatory/prothrombotic molecules, such as VCAM-1. In fibroblasts, AGE-RAGE interaction modulates the production of collagen. In smooth muscle cells, AGE-RAGE interaction modulates the migration, proliferation, and expression of matrix modifying molecules. In mononuclear phagocytes, AGE-RAGE interaction modulates chemotaxis and haptotaxis and the expression of proinflammatory/prothrombotic molecules. In lymphocytes, AGE-RAGE interaction stimulates the proliferation and generation of interleukin-2.

The AGE-RAGE interaction can mediate a vicious cycle of cellular perturbation and tissue injury with implication for aging, inflammation, neurodegeneration, and diabetic complications. Specific consequences of AGE accumulation are the up-regulation of RAGE itself, and the attraction of inflammatory cells, such as polymorphonuclear leukocytes, mononuclear phagocytes, and lymphocytes. Such inflammatory cells, normally mediating homeostatic mechanisms, such as removal of infections substances or necrotic debris, take on new roles in this inflammatory cascade. For example, release of S100 calgranulins and/or amphoterin from such cells triggers a new wave of inflammatory and cell stress reactions. In an autocrine and/or paracrine manner, engagement of these species with RAGE generates another wave of cell perturbing substances. One consequence of ligand-RAGE interaction is the further generation or reactive oxygen species (ROS), which may beget further AGE generation, inflammation, and ROS production. This can feed back to sustain the cycle of stress in a wide range of cell types, thus eventually causing tissue dysfunction and irreparable damage.

The co-localization of RAGE and amphoterin at the leading edge of advancing neurites indicates a potential contribution to cellular migration, and in pathologies such as tumor invasion. In this regard, blockade of RAGE-amphoterin has been shown to decrease growth and metastases of both implanted tumors and tumors developing spontaneously. Inhibition of the RAGE-amphoterin interaction has specifically been shown to suppress activation of p44/p42, p38 and SAP/JNK MAP kinases, and molecular effector mechanisms importantly linked to tumor proliferation, invasion and expression of matrix metalloproteinases.

The binding of S100 calgranulins with RAGE is particularly implicated in triggering extracellular signaling pathways, thereby amplifying inflammation. S100 calgranulins are abundant in the joints of arthritis patients, and their binding to RAGE is strongly linked to rheumatoid arthritis. RAGE-S100 calgranulin interaction has been shown to increase the severity of joint inflammation and bone damage. Moreover, blockade of RAGE-S100 calgranulin binding in arthritic mouse models has shown that joints so treated produced fewer inflammatory molecules, had less swelling and fewer deformities, and suffered less bone and cartilage destruction than controls.

The ability to inhibit interaction of signaling of RAGE and the various ligands described herein, the present invention allows for treatment of multiple conditions by inhibiting activation or expression of various enzymes and pathways, the expression or activation of which are known to be associated with undesirable conditions. For example, blockade of RAGE-ligand interaction by 2-O desulfated heparin will prevent pro-inflammatory signaling by the RAGE receptor. Signaling cascades activated upon ligand-RAGE interaction include pathways, such as p21^{ras}, ERK 1/2 (p44/p42) MAP kinases, p38 and SAPK/JNK MAP kinases, rho GTPases, phosphoinositol-3 kinase, and JAK/STAT, as well as activation of the transcription factors NF-κB and cAmp response element binding protein (CREB). Blockade of RAGE-ligand interaction by 2-O desulfated heparin will also prevent RAGE-mediated production of pro-inflammatory cytokines such as tumor necrosis factor-α (TNF-α), interleukin-1 (IL-1), IL-6, IL-8, granulocyte-macrophage colony stimulating factor (GMCSF), inducible nitric oxide synthase (iNOS), reduce RAGE-mediated expression of integrins such as ICAM-1, E-selectin and VCAM-1, and reduce RAGE-mediated expression of pro-angiogenesis proteins such as vascular endothelial growth factor (VEGF). By blocking RAGE-ligand interaction with Mac-1 (CD11b/CD18), 2-O desulfated heparin will reduce influx of inflammatory cells such as polymorphonuclear neutrophils (PMNs) and monocytes into inflamed tissue, thereby reducing secondary magnification of inflammation by these cell types. By blocking RAGE-ligand interaction with Mac-1 (CD11b/CD18), 2-O desulfated heparin will also prevent RAGE-mediated activation of PMNs, circulating monocytes and tissue monocyte-macrophages such as alveolar macrophages, reducing the pro-inflammatory and pro-fibrotic activities of these cell types to mediate tissue injury, tissue fibrosis and failure of the inflamed and fibrotic organ in which RAGE is activated.

In light of the ability to generally block interaction or signaling of RAGE and its whole range of ligands, the methods of the present invention are clearly capable of providing for treatment of a wide variety of diseases and conditions. In fact, any disease or condition linked to interaction or signaling of RAGE and its ligands can be treated according to the present invention. In particular, the present invention provides for the treatment of conditions such as diabetes, inflammation, renal failure, aging, systemic amyloidosis, Alzheimer's disease, inflammatory arthritis, atherosclerosis, colitis, periodontal diseases, psoriasis, atopic dermatitis, rosacea, multiple sclerosis, chronic obstructive pulmonary disease (COPD), cystic fibrosis, photoaging of the skin, age-related macular degeneration, and acute lung injury.

Accumulation of AGEs in extracellular matrix proteins is typically part of the physiological process of aging; however, this accumulation happens earlier, and with an accelerated rate in diabetes mellitus than in non-diabetic individuals. Enhanced RAGE expression in human diabetic atherosclerotic plaques has been shown to co-localize with COX-2, type 1/type 2 microsomal Prostaglandin E₂, and matrix metalloproteinases, particularly in macrophages at the vulnerable regions of the atherosclerotic plaques. Blockade of the interaction of AGEs with RAGE, such as by 2-0 desulfated heparin, can be effective for treating many complications typically associated with diabetes. For example, blockade of RAGE ligation by AGEs can prevent signaling of RAGE-related expression of the growth factor transforming growth factor-beta 1, which mediates diabetes related renal failure (Ceol M, et al. J Am Soc Nephrol 2000; 11: 2324-2326). Inhibition of RAGE ligation by diabetes-related AGE-products can also decrease the RAGE-related production of vascular endothelial growth factor (VEGF), thereby preventing development of endothelial overgrowth that causes proliferative diabetic retinopathy and blindness complicating diabetes. By inhibiting interaction of diabetes-related AGE-products with RAGE, 2-O desulfated heparin can also decrease RAGE-related diabetic neuropathic changes leading to diabetes-related neuropathies.

Blocking the interaction between RAGE and its other ligands is also effective for treatment of other undesirable health conditions. For example, RAGE serves as a cell surface receptor for Amyloid β peptide (Aβ), a cleavage product of the β-amyloid precursor protein which accumulates in Alzheimer's disease and β sheet fibrils. RAGE is expressed at increased levels in cells in the brains of Alzheimer's patients, including neurons and cerebral blood vessels (endothelial cells and smooth muscle cells). When fibrils of Aβ bind to RAGE-bearing cells, their functional properties can become distorted. Such altered function can have multiple consequences including decreased cerebral blood flow and diminished synaptic plasticity, ultimately leading to neuronal dysfunction underlying dementia. In Alzheimer's disease, RAGE ligation by the Alzheimer's β-peptidecan specifically initiate the process of neuronal cell death, which is characteristic of the Alzheimer's dementia process.

RAGE blockade can also affect systemic amyloidosis processes. Deposition of amyloid in tissues displaces normal structures and, at high concentrations, can exert nonspecific toxic effects on cells by disturbing the integrity of membranes. Amyloid deposits and low-molecular weight amyloid fragments are believed to be biologically active via their interaction with specific cell surface receptors that appear to act early in the disease process when the amyloid burden is low, possibly by amplifying the response to nascent amyloid. RAGE binds β-sheet fibrillar material regardless of the composition of the subunits (amyloid-β peptide, Aβ, amylin, serum amyloid A, and prion-derived peptides, among others), and deposition of amyloid results in enhanced expression of RAGE. For example, in the brains of patients with Alzheimer disease, RAGE expression increases in neurons and glia. The consequences of Aβ ligation of RAGE appear to be quite different on neurons versus microglia. Whereas microglia become activated as a consequence of Aβ-RAGE interaction, as reflected by increased motility and expression of cytokines, early RAGE-mediated neuronal activation is superseded by cytotoxicity at later times. Inhibition of Aβ-induced cerebral vasoconstriction and reduced transfer of the amyloid peptide across the blood-brain barrier following receptor blockade provide further evidence of a role for RAGE in cellular interactions with Aβ.

Ligation of RAGE by amyloid proteins initiates the inflammatory change leading to organ failures, including neuropathies, renal, pulmonary and hepatic failure characteristic of systemic amyloidosis. In vivo, blockade of RAGE in a murine model of systemic amyloidosis suppressed Amyloid-induced nuclear translocation of NF-kB and cellular activation (Yan, S D, et al., Nature Medicine, 2000, 6, 643-651).

RAGE is also a signal transduction receptor for members of the S100 calgranulin family of proinflammatory cytokines (including ENRAGEs). This family is comprised of closely-related polypeptides released from activated inflammatory cells, including polymorphonuclear leukocytes, peripheral blood-derived mononuclear phagocytes and lymphocytes. These proinflammatory cytokines are known to accumulate at sites of chronic inflammation, such as psoriatic skin disease, cystic fibrosis, inflammatory bowel disease, and rheumatoid arthritis. Ligation of RAGE by ENRAGEs has been shown to mediate activation of endothelial ells, macrophages, and lymphocytes. RAGE ligation can also be linked to further proinflammatory conditions, such as inflammatory arthritis, atherosclerosis, colitis, psoriasis, atopic dermatitis, and can further arise from ligation by AGE products formed by the oxidative effects of phagocytes. In these conditions, RAGE ligation produces a secondary wave of inflammation that magnifies the original, initiating inflammatory response, perpetuating the original pathophysiologic process that produced the inflammatory condition. In vivo, blockade of RAGE has been shown to suppress inflammation in murine models of delayed-type hypersensitivity and inflammatory bowel disease. In parallel with suppression of the inflammatory phenotype, inhibition of RAGE-S100 calgranulin interaction has been shown to decrease NF-kB activation and expression of proinflammatory cytokines in tissues, indicating receptor blockage changed the course of the inflammatory response.

In conditions characterized by increased accumulation and expression of RAGE and its ligands, such as diabetic atherosclerotic lesions and periodontium, chronic disorders such as rheumatoid arthritis and inflammatory bowel disease, and Alzheimer's disease, enhanced inflammatory responses have been linked to ongoing cellular perturbation. One consequence of ligand-RAGE-mediated activation MAP kinases and NF-kB is increased transcription and translation of vascular cell adhesion molecule (VCAM-1). At the cell surface, endothelium stimulated by a range of mediators, such as endotoxin, tumor necrosis factor α (TNF α), and AGEs, display increased adhesion of proinflammatory mononuclear cells via VCAM-1. Evidence also indicates that the proinflammatory effects of VCAM-1 are not limited to cellular adhesion events, as binding of ligand to VCAM-1 in endothelial cell lines and primary cultures induced activation of endothelial NADPH oxidase, a process shown to be essential for lymphocyte migration through the stimulated cells. This indicates that activation of RAGE at the cell surface may initiate a cascade of events including activation of NADPH oxidase and a range of proinflammatory mediators, such as VCAM-1.

As RAGE has been indicated as a receptor for amphoterin, a molecule linked to neurite outgrowth in developing neurons of the central and peripheral nervous system, the amphoterin-RAGE interaction can be linked to cellular migration and invasiveness. For example, the expression of amphoterin and RAGE has been shown to be increased in tumors. Thus, blockade of RAGE in vivo can suppress local growth and distant spread of tumors forming endogenously. Moreover, certain S100s, such as S100B, are linked to nervous system stress, and other, such as S100P, are linked to cancer. In this context, RAGE-dependent ligation of S100P has been shown to increase the proliferation and survival of cancer cells in vitro. In further relation, blockade of RAGE signaling on amphoterin-coated matrices can suppress activation of p44/42, p38, and SAPK/JNK kinases.

One surprising aspect of the present invention is the ability to provide a single compound capable of effecting treatment in a variety of conditions related to RAGE ligation. As previously pointed out, there is much confusion in the art as to the mode of interaction between RAGE and its ligands. Ionic charge, molecule size, molecule shape, and attached side groups have all been implicated as playing a part in RAGE ligation. The present invention, however, allows for the use of a single compound, such as 2-O, 3-O desulfated heparin, to inhibit the whole range of the RAGE ligands. In other words, the compounds of the invention are not limited by their specific charge, a specific shape, or the presence of a specific side group to interact with RAGE. Rather, the compounds of the invention will interact with RAGE to block its further interaction or signaling with the whole range of known RAGE ligands.

This ability is illustrated below in the Examples showing empirical experimentation with a variety of desulfated and carboxyl reduced heparins to determine their ability to inhibit RAGE-ligand activity, using Mac-1 (CD11b/CD18)-mediated attachment of U937 human monocytes to immobilized RAGE as a paradigm RAGE-ligand interaction. Those examples show wide and surprising differences in the requirement of various heparin side groups and heparin sizes for inhibition of ligand-RAGE interaction.

Biologically active variants of 2-O desulfated heparin are particularly also encompassed by the invention. Such variants should retain the activity of the original compound as a RAGE ligation inhibitor; however, the presence of additional activities would not necessarily limit the use thereof in the present invention.

According to one embodiment of the invention, suitable biologically active variants comprise analogues and derivatives of the compounds described herein. Indeed, a single compound, such as those described herein, may give rise to an entire family of analogues or derivatives having similar activity and, therefore, usefulness according to the present invention. Likewise, a single compound, such as those described herein, may represent a single family member of a greater class of compounds useful according to the present invention. Accordingly, the present invention fully encompasses not only the compounds described herein, but analogues and derivatives of such compounds, particularly those identifiable by methods commonly known in the art and recognizable to the skilled artisan. An analog is defined as a substitution of an atom or functional group in the heparin molecule with a different atom or functional group that usually has similar properties. A derivative is defined as an O-desulfated heparin that has another molecule or atom attached to it.

In certain embodiments, an analog of 2-O desulfated heparin, as described herein, includes compounds having the same functions as 2-O desulfated heparin for use in the methods of the invention (including minimal anticoagulant activity), and specifically includes homologs that retain these functions. For example, various substituents on the heparin polymer can be removed or altered by any of many means known to those skilled in the art, such as acetylation, deacetylation, decarboxylation, oxidation, etc., so long as such alteration or removal does not substantially increase the low anticoagulation activity of the 2-O desulfated heparin. Any analog can be readily assessed for these activities by known methods given the teachings herein.

The 2-O desulfated heparin of the invention may particularly include 2-O desulfated heparin having modifications, such as reduced molecular weight or acetylation, deacetylation, oxidation, and decarboxylation, as long as it retains its ability to function according to the methods of the invention. Such modifications can be made either prior to or after partial desulfation and methods for modification are standard in the art. As noted above, 2-O desulfated heparin can particularly be modified to have a reduced molecular weight, and several low molecular weight modifications of heparin have been developed (see page 581, Table 27.1 Heparin, Lane & Lindall).

Periodate oxidation (U.S. Pat. No. 5,250,519, which is incorporated herein by reference) is one example of a known oxidation method that produces an oxidized heparin having reduced anticoagulant activity. Other oxidation methods, also well known in the art, can be used. Additionally, for example, decarboxylation of heparin is also known to decrease anticoagulant activity, and such methods are standard in the art. Furthermore, some low molecular weight heparins are known in the art to have decreased anti-coagulant activity, including Vasoflux, a low molecular weight heparin produced by nitrous acid depolymerization, followed by periodate oxidation (Weitz J I, Young E, Johnston M, Stafford A R, Fredenburgh J C, Hirsh J. Circulation. 99:682-689, 1999). Thus, modified O-desulfated heparin (or heparin analogs or derivatives) contemplated for use in the present invention can include, for example, periodate-oxidized 2-O desulfated heparin, decarboxylated 2-O desulfated heparin, acetylated 2-O desulfated heparin, deacetylated 2-O desulfated heparin, deacetylated, oxidized 2-O desulfated heparin, and low molecular weight 2-O desulfated heparin.

The 2-O desulfated heparin used according to the present invention can be in any form useful for delivery to a patient provided the 2-O desulfated heparin maintains the activity useful in the methods of the invention, particularly the low anticoagulation activity of the 2-O desulfated heparin. Non-limiting examples of further forms the 2-O desulfated heparin may take on that are encompassed by the invention include esters, amides, salts, solvates, prodrugs, or metabolites. Such further forms may be prepared according to methods generally known in the art, such as, for example, those methods described by J. March, Advanced Organic Chemistry: Reactions, Mechanisms and Structure, 4^{th} Ed. (New York: Wiley-Interscience, 1992), which is incorporated herein by reference.

In the case of solid compositions, it is understood that the compounds used in the methods of the invention may exist in different forms. For example, the compounds may exist in stable and metastable crystalline forms and isotropic and amorphous forms, all of which are intended to be within the scope of the present invention.

While it is possible for the sulfated polysaccharides (such as 2-O desulfated heparin) used in the methods of the present invention to be administered in the raw chemical form, it is preferred for the compounds to be delivered as a pharmaceutical composition. Accordingly, there are provided by the present invention pharmaceutical compositions comprising 2-O desulfated heparin or other sulfated polysaccharides. As such, the compositions used in the methods of the present invention comprise sulfated polysaccharides or pharmaceutically acceptable variants thereof.

The sulfated polysaccharides can be prepared and delivered together with one or more pharmaceutically acceptable carriers therefore, and optionally, other therapeutic ingredients. Carriers should be acceptable in that they are compatible with any other ingredients of the composition and not harmful to the recipient thereof. Such carriers are known in the art. See, Wang et al. (1980) J. Parent. Drug Assn. 34(6):452-462, herein incorporated by reference in its entirety.

Compositions may include short-term, rapid-onset, rapid-offset, controlled release, sustained release, delayed release, and pulsatile release compositions, providing the compositions achieve administration of a compound as described herein. See Remington's Pharmaceutical Sciences (18^{th} ed.; Mack Publishing Company, Eaton, Pa., 1990), herein incorporated by reference in its entirety.

Pharmaceutical compositions for use in the methods of the invention are suitable for various modes of delivery, including oral, parenteral, and topical (including dermal, buccal, and sublingual) administration. Administration can also be via nasal spray, surgical implant, internal surgical paint, infusion pump, or other delivery device. The most useful and/or beneficial mode of administration can vary, especially depending upon the condition of the recipient. In preferred embodiments, the compositions of the invention are administered intravenously, subcutaneously, or by inhalation. When provided as an inhaled aerosol for intrapulmonary delivery, the micronized particles are preferably less than 10 microns (micrometers) and most preferable less than 5 microns in diameter. For delivery into the airway or lung, sulfated polysaccharides can be delivered as a micronized powder or inhaled as a solution with the use of a commercially available nebulizer device. For delivery to the nasal mucosa, sulfated polysaccharides can be administered as a solution that is aerosolized by a commercially available misting or spray device, or it can be delivered as a nasally administered micronized dry powder.

The pharmaceutical compositions may be conveniently made available in a unit dosage form, whereby such compositions may be prepared by any of the methods generally known in the pharmaceutical arts. Generally speaking, such methods of preparation comprise combining (by various methods) the sulfated polysaccharides with a suitable carrier or other adjuvant, which may consist of one or more ingredients. The combination of the sulfated polysaccharides with the one or more adjuvants is then physically treated to present the composition in a suitable form for delivery (e.g., shaping into a tablet or forming an aqueous suspension).

Pharmaceutical compositions suitable for oral dosage may take various forms, such as tablets, capsules, caplets, and wafers (including rapidly dissolving or effervescing), each containing a predetermined amount of the sulfated polysaccharides. The compositions may also be in the form of a powder or granules, a solution or suspension in an aqueous or non-aqueous liquid, and as a liquid emulsion (oil-in-water and water-in-oil). The sulfated polysaccharides may also be delivered as a bolus, electuary, or paste. It is generally understood that methods of preparations of the above dosage forms are generally known in the art, and any such method would be suitable for the preparation of the respective dosage forms for use in delivery of the compositions according to the present invention.

In one embodiment, sulfated polysaccharides may be administered orally in combination with a pharmaceutically acceptable vehicle such as an inert diluent or an edible carrier. Oral compositions may be enclosed in hard or soft shell gelatin capsules, may be compressed into tablets or may be incorporated directly with the food of the patient's diet. The percentage of the composition and preparations may be varied; however, the amount of substance in such therapeutically useful compositions is preferably such that an effective dosage level will be obtained. To enhance oral penetration and gastrointestinal absorption, sulfated polysaccharides can be formulated with mixtures of olive oil, bile salts, or sodium N-[8-(2-hydroxybenzoyl)amino]caprylate (SNAC). A preferred ratio of about 2.25 g of SNAC to 200 to 1,000 mg 2-O desulfated heparin is employed. Additional formulations that facilitate gastrointestinal absorption can be made by formulating phospholipids-cation-precipitate cochleate delivery vesicles of 2-O desulfated heparin with phosphotidylserine and calcium, using methods such as described in U.S. Pat. Nos. 6,153,217; 5,994,318 and 5,840,707, which are incorporated herein by reference.

Hard capsules containing the sulfated polysaccharides may be made using a physiologically degradable composition, such as gelatin. Such hard capsules comprise the sulfated polysaccharides, and may further comprise additional ingredients including, for example, an inert solid diluent such as calcium carbonate, calcium phosphate, or kaolin. Soft gelatin capsules containing the compound may be made using a physiologically degradable composition, such as gelatin. Such soft capsules comprise the compound, which may be mixed with water or an oil medium such as peanut oil, liquid paraffin, or olive oil.

Sublingual tablets are designed to dissolve very rapidly. Examples of such compositions include ergotamine tartrate, isosorbide dinitrate, and isoproterenol HCL. The compositions of these tablets contain, in addition to the drug, various soluble excipients, such as lactose, powdered sucrose, dextrose, and mannitol. The solid dosage forms of the present invention may optionally be coated, and examples of suitable coating materials include, but are not limited to, cellulose polymers (such as cellulose acetate phthalate, hydroxypropyl cellulose, hydroxypropyl methylcellulose, hydroxypropyl methylcellulose phthalate, and hydroxypropyl methylcellulose acetate succinate), polyvinyl acetate phthalate, acrylic acid polymers and copolymers, and methacrylic resins (such as those commercially available under the trade name EUDRAGIT®), zein, shellac, and polysaccharides.

Powdered and granular compositions of a pharmaceutical preparation may be prepared using known methods. Such compositions may be administered directly to a patient or used in the preparation of further dosage forms, such as to form tablets, fill capsules, or prepare an aqueous or oily suspension or solution by addition of an aqueous or oily vehicle thereto. Each of these compositions may further comprise one or more additives, such as dispersing or wetting agents, suspending agents, and preservatives. Additional excipients (e.g., fillers, sweeteners, flavoring, or coloring agents) may also be included in these compositions.

Liquid compositions of pharmaceutical compositions which are suitable for oral administration may be prepared, packaged, and sold either in liquid form or in the form of a dry product intended for reconstitution with water or another suitable vehicle prior to use.

A tablet containing sulfated polysaccharides may be manufactured by any standard process readily known to one of skill in the art, such as, for example, by compression or molding, optionally with one or more adjuvant or accessory ingredient. The tablets may optionally be coated or scored and may be formulated so as to provide slow or controlled release of the sulfated polysaccharides.

Adjuvants or accessory ingredients for use in the compositions can include any pharmaceutical ingredient commonly deemed acceptable in the art, such as binders, fillers, lubricants, disintegrants, diluents, surfactants, stabilizers, preservatives, flavoring and coloring agents, and the like. Binders are generally used to facilitate cohesiveness of the tablet and ensure the tablet remains intact after compression. Suitable binders include, but are not limited to: starch, polysaccharides, gelatin, polyethylene glycol, propylene glycol, waxes, and natural and synthetic gums. Acceptable fillers include silicon dioxide, titanium dioxide, alumina, talc, kaolin, powdered cellulose, and microcrystalline cellulose, as well as soluble materials, such as mannitol, urea, sucrose, lactose, dextrose, sodium chloride, and sorbitol. Lubricants are useful for facilitating tablet manufacture and include vegetable oils, glycerin, magnesium stearate, calcium stearate, and stearic acid. Disintegrants, which are useful for facilitating disintegration of the tablet, generally include starches, clays, celluloses, algins, gums, and crosslinked polymers. Diluents, which are generally included to provide bulk to the tablet, may include dicalcium phosphate, calcium sulfate, lactose, cellulose, kaolin, mannitol, sodium chloride, dry starch, and powdered sugar. Surfactants suitable for use in the composition according to the present invention may be anionic, cationic, amphoteric, or nonionic surface active agents. Stabilizers may be included in the compositions to inhibit or lessen reactions leading to decomposition of the sulfated polysaccharides, such as oxidative reactions.

Solid dosage forms may be formulated so as to provide a delayed release of the sulfated polysaccharides, such as by application of a coating. Delayed release coatings are known in the art, and dosage forms containing such may be prepared by any known suitable method. Such methods generally include that, after preparation of the solid dosage form (e.g., a tablet or caplet), a delayed release coating composition is applied. Application can be by methods, such as airless spraying, fluidized bed coating, use of a coating pan, or the like. Materials for use as a delayed release coating can be polymeric in nature, such as cellulosic material (e.g., cellulose butyrate phthalate, hydroxypropyl methylcellulose phthalate, and carboxymethyl ethylcellulose), and polymers and copolymers of acrylic acid, methacrylic acid, and esters thereof.

Solid dosage forms according to the present invention may also be sustained release (i.e., releasing the sulfated polysaccharides over a prolonged period of time), and may or may not also be delayed release. Sustained release compositions are known in the art and are generally prepared by dispersing a drug within a matrix of a gradually degradable or hydrolyzable material, such as an insoluble plastic, a hydrophilic polymer, or a fatty compound. Alternatively, a solid dosage form may be coated with such a material.

Compositions for parenteral administration include aqueous and non-aqueous sterile injection solutions, which may further contain additional agents, such as anti-oxidants, buffers, bacteriostats, and solutes, which render the compositions isotonic with the blood of the intended recipient. The compositions may include aqueous and non-aqueous sterile suspensions, which contain suspending agents and thickening agents. Such compositions for parenteral administration may be presented in unit-dose or multi-dose containers, such as, for example, sealed ampoules and vials, and may be stores in a freeze-dried (lyophilized) condition requiring only the addition of the sterile liquid carrier, for example, water (for injection), immediately prior to use. Extemporaneous injection solutions and suspensions may be prepared from sterile powders, granules, and tablets of the kind previously described.

The compositions for use in the methods of the present invention may also be administered transdermally, wherein the sulfated polysaccharide is incorporated into a laminated structure (generally referred to as a “patch”) that is adapted to remain in intimate contact with the epidermis of the recipient for a prolonged period of time. Typically, such patches are available as single layer “drug-in-adhesive” patches or as multi-layer patches where the active agents are contained in a layer separate from the adhesive layer. Both types of patches also generally contain a backing layer and a liner that is removed prior to attachment to the skin of the recipient. Transdermal drug delivery patches may also be comprised of a reservoir underlying the backing layer that is separated from the skin of the recipient by a semi-permeable membrane and adhesive layer. Transdermal drug delivery may occur through passive diffusion or may be facilitated using electrotransport or iontophoresis.

Compositions for rectal delivery include rectal suppositories, creams, ointments, and liquids. Suppositories may be presented as the sulfated polysaccharide in combination with a carrier generally known in the art, such as polyethylene glycol. Such dosage forms may be designed to disintegrate rapidly or over an extended period of time, and the time to complete disintegration can range from a short time, such as about 10 minutes, to an extended period of time, such as about 6 hours.

Topical compositions may be in any form suitable and readily known in the art for delivery of active agents to the body surface, including dermally, buccally, and sublingually. Typical examples of topical compositions include ointments, creams, gels, pastes, and solutions. Compositions for topical administration in the mouth also include lozenges.

In certain embodiments, the compounds and compositions disclosed herein can be delivered via a medical device. Such delivery can generally be via any insertable or implantable medical device, including, but not limited to stents, catheters, balloon catheters, shunts, or coils. In one embodiment, the present invention provides medical devices, such as stents, the surface of which is coated with a compound or composition as described herein. The medical device of this invention can be used, for example, in any application for treating, preventing, or otherwise affecting the course of a disease or condition, such as those disclosed herein.

In another embodiment of the invention, pharmaceutical compositions comprising sulfated polysaccharides are administered intermittently. Administration of the therapeutically effective dose may be achieved in a continuous manner, as for example with a sustained-release composition, or it may be achieved according to a desired daily dosage regimen, as for example with one, two, three, or more administrations per day. By “time period of discontinuance” is intended a discontinuing of the continuous sustained-released or daily administration of the composition. The time period of discontinuance may be longer or shorter than the period of continuous sustained-release or daily administration. During the time period of discontinuance, the level of the components of the composition in the relevant tissue is substantially below the maximum level obtained during the treatment. The preferred length of the discontinuance period depends on the concentration of the effective dose and the form of composition used. The discontinuance period can be at least 2 days, at least 4 days or at least 1 week. In other embodiments, the period of discontinuance is at least 1 month, 2 months, 3 months, 4 months or greater. When a sustained-release composition is used, the discontinuance period must be extended to account for the greater residence time of the composition in the body. Alternatively, the frequency of administration of the effective dose of the sustained-release composition can be decreased accordingly. An intermittent schedule of administration of a composition of the invention can continue until the desired therapeutic effect, and ultimately treatment of the disease or disorder, is achieved.

Administration of the composition comprises administering sulfated polysaccharides in combination with one or more further pharmaceutically active agents (i.e., co-administration). Accordingly, it is recognized that the pharmaceutically active agents described herein can be administered in a fixed combination (i.e., a single pharmaceutical composition that contains both active agents). Alternatively, the pharmaceutically active agents may be administered simultaneously (i.e., separate compositions administered at the same time). In another embodiment, the pharmaceutically active agents are administered sequentially (i.e., administration of one or more pharmaceutically active agents followed by separate administration or one or more pharmaceutically active agents). One of skill in the art will recognized that the most preferred method of administration will allow the desired therapeutic effect.

Delivery of a therapeutically effective amount of a composition according to the invention may be obtained via administration of a therapeutically effective dose of the composition. Accordingly, in one embodiment, a therapeutically effective amount is an amount effective to inhibit ligation of RAGE by one or more ligands, and in certain embodiments the level of inhibition is sufficient to reduce or eliminate the negative biological implications of a condition, such as by reducing the severity of or the elimination of symptoms associated with the condition.

The concentration of sulfated polysaccharides in the composition will depend on absorption, inactivation, and excretion rates of the sulfated polysaccharides as well as other factors known to those of skill in the art. It is to be noted that dosage values will also vary with the severity of the condition to be alleviated. It is to be further understood that for any particular subject, specific dosage regimens should be adjusted over time according to the individual need and the professional judgment of the person administering or supervising the administration of the compositions, and that the dosage ranges set forth herein are exemplary only and are not intended to limit the scope or practice of the claimed composition. The active ingredient may be administered at once, or may be divided into a number of smaller doses to be administered at varying intervals of time.

It is contemplated that compositions of the invention comprising one or more active agents described herein will be administered in therapeutically effective amounts to a mammal, preferably a human. An effective dose of a compound or composition for treatment of any of the conditions or diseases described herein can be readily determined by the use of conventional techniques and by observing results obtained under analogous circumstances. The effective amount of the compositions would be expected to vary according to the weight, sex, age, and medical history of the subject. Of course, other factors could also influence the effective amount of the composition to be delivered, including, but not limited to, the specific disease involved, the degree of involvement or the severity of the disease, the response of the individual patient, the particular compound administered, the mode of administration, the bioavailability characteristics of the preparation administered, the dose regimen selected, and the use of concomitant medication. The compound is preferentially administered for a sufficient time period to alleviate the undesired symptoms and the clinical signs associated with the condition being treated. Methods to determine efficacy and dosage are known to those skilled in the art. See, for example, Isselbacher et al. (1996) Harrison's Principles of Internal Medicine 13 ed., 1814-1882, herein incorporated by reference.

In certain embodiments, the 2-O desulfated heparin provided according to the invention preferably comprises a dose of about 0.1 mg/kg patient body weight to about 100 mg/kg. In further embodiments, the medicament comprises a dose of about 0.2 mg/kg to about 90 mg/kg, about 0.3 mg/kg to about 80 mg/kg, about 0.4 mg/kg to about 70 mg/kg, about 0.5 mg/kg to about 60 mg/kg, about 0.5 mg/kg to about 50 mg/kg, about 1 mg/kg to about 50 mg/kg, about 2 mg/kg to about 50 mg/kg, or about 3 mg/kg to about 25 mg/kg patient body weight.

### EXAMPLES

The present invention is more particularly described in the following examples which are intended as illustrative only. Numerous modifications and variations therein will be apparent to those skilled in the art.

### Example 1

### Production of Nonanticoagulant 2-O Desulfated Heparin

Partially desulfated 2-O desulfated heparin (ODS heparin) was produced in commercially practical quantities by methods described in U.S. Pat. No. 5,668,188; U.S. Pat. No. 5,912,237; and U.S. Pat. No. 6,489,311. Modification to ODS heparin was made by adding 500 gm of porcine intestinal mucosal sodium heparin from lot EM3037991 to 10 L (liters) deionized water (5% by weight final heparin concentration). Sodium borohydride was added to achieve 1% final concentration and the mixture was incubated overnight at 25° C. Sodium hydroxide was then added to achieve 0.4 M final concentration (pH greater than 13) and the mixture was lyophilized to dryness. Excess sodium borohydride and sodium hydroxide were removed by ultrafiltration. The final product was adjusted to pH 7.0, precipitated by the addition of three volumes of cold ethanol and then dried. The 2-O desulfated heparin produced by this procedure was a fine crystalline slightly off-white powder with less than 10 USP units/mg anticoagulant activity and less than 10 anti Xa units/mg anticoagulant activity. The structure of this heparin is shown in FIG. 1. Molecular weight was determined by high performance size exclusion chromatography in conjunction with multiangle laser light scattering, using a miniDAWN detector (Wyatt Technology Corporation, Santa Barbara, Calif.) operating at 690 nm (nanometers). Compared with an average molecular weight of 13.1 kD for the starting material, ODS Heparin had an average molecular weight of 11.8 kD.

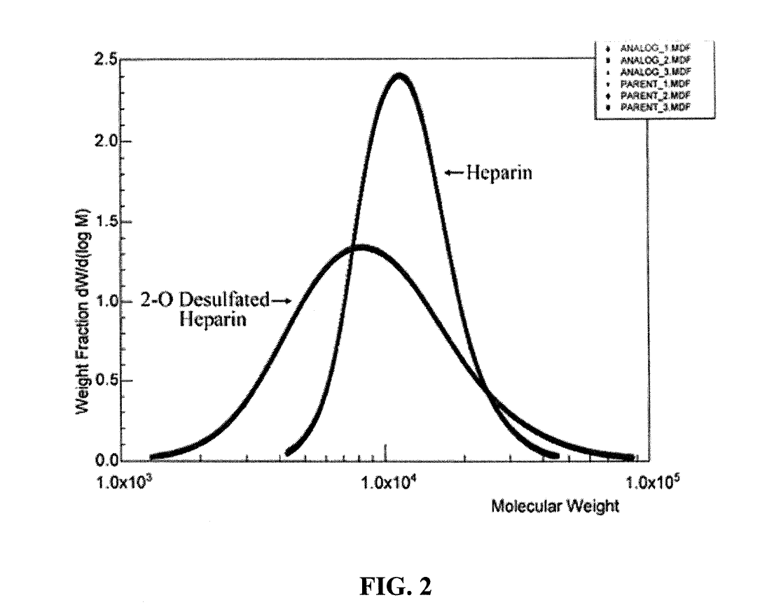

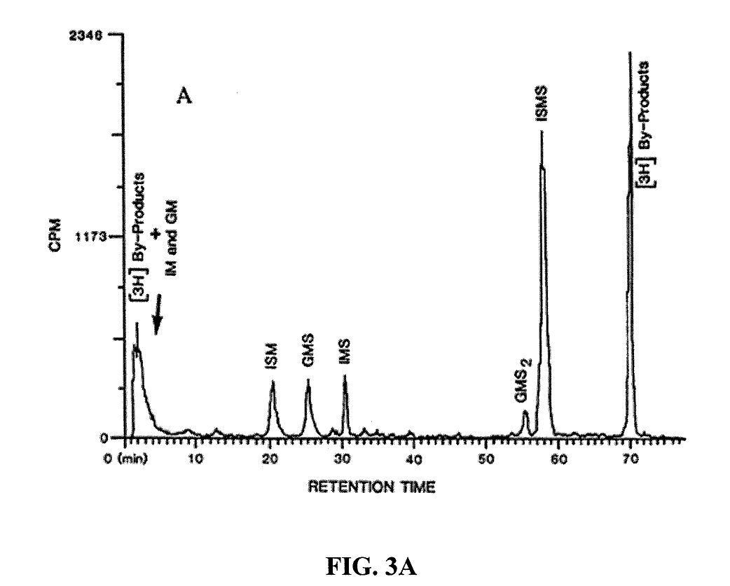

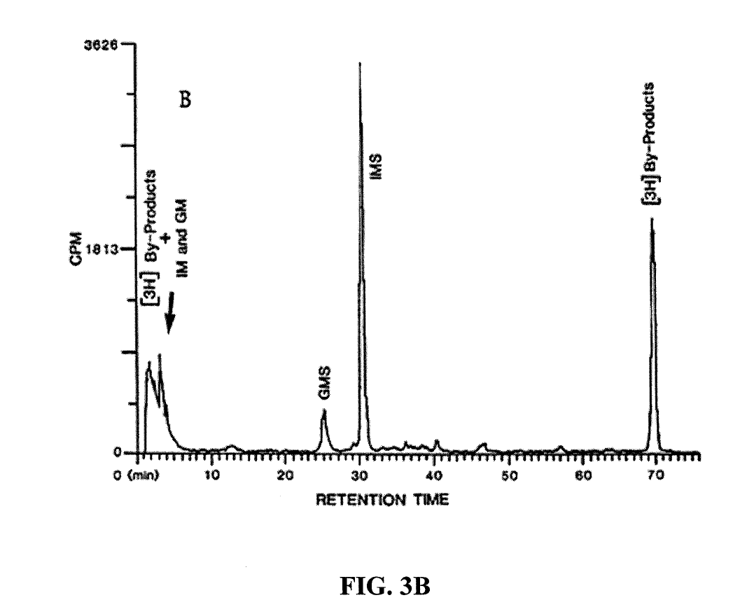

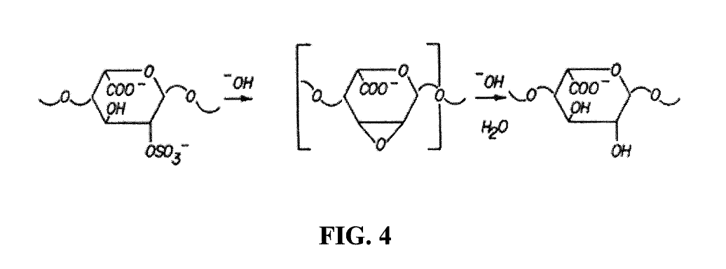

Provided in FIG. 2 are the differential molecular weight distributions of the parent molecule and ODS heparin. Disaccharide analysis was performed by the method of Guo and Conrad (Anal Biochem 1988; 178:54-62). Compared to the starting material shown in FIG. 3A, ODS heparin was a 2-O desulfated heparin (shown in FIG. 3B) characterized by conversion of ISM [L-iduronic acid(2-sulfate)-2,5-anhydromannitol] to IM [L-iduronic acid-2,5-anhydromannitol], and ISMS [L-iduronic acid(2-sulfate)-2,5 anhydromannitol(6-sulfate)] to IMS L-iduronic acid-2,5-anhydromannitol(6-sulfate), both indicating 2-O desulfation. The proposed sequence of 2-O desulfation is shown in FIG. 4. ODS heparin was also a 3-O desulfated heparin, characterized by conversion of GMS2 [D47 glucuronic acid-2,5-anhydromannitol(3,6-disulfate)] to GMS [D-glucuronic acid-2,5-anhydromannitol(6-sulfate)], indicating 3-O desulfation.

The potential of this 2-O, 3-O desulfated heparin (ODSH) to interact with HIT antibody and active platelets was studied using donor platelets and serum from three different patients clinically diagnosed with HIT-2, by manifesting thrombocytopenia related to heparin exposure, correction of thrombocytopenia with removal of heparin, and a positive platelet activation test, with or without thrombosis. Two techniques were employed to measure platelet activation in response to heparin or 2-O desulfated heparin in the presence of HIT-reactive serum.

The first technique was the serotonin release assay (SRA), considered the gold standard laboratory test for HIT, and performed as described by Sheridan D, et al., Blood 1986; 67:27-30. Washed platelets were loaded with ¹⁴C serotonin (¹⁴C-hydroxy-tryptamine-creatine sulfate, Amersham), and then incubated with various concentrations of test heparin or heparin analog in the presence of serum from known HIT-positive patients as a source of antibody. Activation was assessed as ¹⁴C serotonin release from platelets during activation, with ¹⁴C serotonin quantitated using a liquid scintillation counter. Formation of the heparin-PF4-HIT antibody complex resulted in platelet activation and isotope release into the buffer medium. Activated platelets are defined as percent isotope release of >20%.

Specifically, using a two-syringe technique, whole blood was drawn from a volunteer donor into sodium citrate (0.109M) at a ratio of 1 part anticoagulant to 9 parts whole blood. The initial 3 ml (milliliters) of whole blood in the first syringe was discarded. The anticoagulated blood was centrifuged (80×g (gravity), 15 min, room temperature) to obtain platelet rich plasma (PRP). The PRP was labeled with 0.1 μCuries ¹⁴-Carbon-serotonin/ml (45 min, 37° C.), then washed and resuspended in albumin-free Tyrode's solution to a count of 300,000 platelets/μl (microliter). HIT serum (20 μl) was incubated (1 hour at room temperature) with 70 μl of the platelet suspension, and 5 μl of 2-O desulfated heparin (0, 0.78, 1.56, 3.13, 6.25, 12.5, 25, 50 and 100 μg (micrograms)/ml final concentrations). For system controls, 10 μl unfractionated heparin (UFH; either 0.1 or 0.5 U/ml final concentrations, corresponding to the concentrations in plasma found in patients on anti-thrombotic or fully anticoagulant doses, respectively) was substituted for the 2-O desulfated heparin in the assay. EDTA was added to stop the reaction, and the mixture was centrifuged to pellet the platelets. ¹⁴C-serotonin released into the supernatant was measured on a scintillation counter. Maximal release was measured following platelet lysis with 10% Triton X-100 (Sigma Chemicals, St. Louis, Mo.). The test was positive if the release was ≧20% serotonin with 0.1 and 0.5 U/ml UFH (no added 2-O desulfated heparin) and <20% serotonin with 100 U/ml UFH. The test was for cross-reactivity of the HIT antibodies with the 2-O desulfated heparin if ≧20% serotonin release occurred.

The second technique was flow cytometric platelet analysis. In this functional test, platelets in whole blood are activated by heparin or heparin analog in the presence of heparin antibody in serum from a patient clinically diagnosed with HIT. Using flow cytometry, platelet activation was determined in two manners: the formation of platelet microparticles and the increase of platelet surface bound P-selectin. Normally, platelets in their unactivated state do not express CD62 on their surface, and platelet microparticles are barely detectable. A positive response is defined as any response significantly greater than the response of the saline control.

Specifically, whole blood drawn by careful double-syringe technique was anticoagulated with hirudin (10 μg/ml final concentration). An aliquot of whole blood (50 μl) was immediately fixed in 1 ml 1% paraformaldehyde (gating control). HIT serum (160 μl) and 2-O desulfated heparin (50 μl; 0, 0.78, 1.56, 3.13, 6.25, 12.5, 25, 50 and 100 μg/ml final concentrations) were added to the whole blood (290 μl) and incubated (37° C., 15 minutes, with stirring at 600 rpm). Aliquots (50 μl) were removed and fixed in 1 ml paraformaldehyde (30 minutes, 4° C.). The samples were centrifuged (350 g, 10 minutes) and the supernatant paraformaldehyde removed. The cells were resuspended in calcium-free Tyrode's solution (500 μl, pH 7.4±0.1). 150 μl cell suspension was added to 6.5 μl fluorescein isothiocyanate (FITC) labeled anti-CD61 antibody (Becton-Dickinson; San Jose, Calif.; specific for GPIIIa on all platelets). Samples were incubated (30 minutes, room temperature) in the dark. All antibodies were titrated against cells expressing their specific antigen prior to experimentation to assess the saturating concentration. Samples were analyzed on an EPICS® XL flow cytometer (Beckman-Couter; Hialeah, Fla.) for forward angle (FALS) and side angle light scatter, and for FITC and PE (phycoerythrin) fluorescence. Prior to running samples each day, a size calibration was made by running fluorescent-labeled beads of known size (Flow-Check; Coulter) and adjusting the gain so that 1.0 μm beads fall at the beginning of the second decade of a 4-decade log FALS light scatter scale. A threshold discriminator set on the FITC signal was used to exclude events not labeled with anti-CD61 antibody (non-platelets).

Using the gating control sample, amorphous regions were drawn to include single platelets and platelet microparticles. Platelet microparticles were distinguished from platelets on the basis of their characteristic flow cytometric profile of cell size (FALS) and FITC fluorescence (CD61 platelet marker). Platelet micro-particles were defined as CD61-positive events that were smaller than the single, nonaggregated platelet population (<˜1 μm). 20,000 total CD61-positive events (platelets) were collected for each sample. Data was reported as a percentage of the total number of CD61-positive events analyzed. In testing for cross-reactivity with a heparin-dependent HIT antibody, the UFH controls (no 2-O desulfated heparin) should show a positive response (increased percentage of CD61 positive events in the platelet microparticle region at 0.1 and 0.5 U/ml UFH, but not at 100 U/ml UFH). The test was positive for cross-reactivity of the HIT antibodies with the 2-O desulfated heparin if an increase in platelet microparticle formation occurred.

The quantitation of P-selectin expression induced on the surface of platelets by HIT-related platelet activation was determined as follows. To quantitate platelet surface expression of P-selection, platelet-rich plasma was collected and platelets were labeled as described above, but additionally labeled with 6.5 μl of phycoerythrin (PE) labeled antibody (Becton-Dickinson; specific for P-selectin expressed on activated platelets). The gating control sample was used to establish the regions of single platelets and platelet microparticles based on FALS and CD61-FITC fluorescence. A histogram of PE fluorescence (P-selectin expression) was gated to exclude platelet aggregates. A marker encompassing the entire peak was set in order to determine the median P-selectin fluorescence. Results were reported in mean fluorescence intensity units (MFI) of CD62 in the non-aggregated platelet population. In testing for cross-reactivity with a heparin-dependent HIT antibody, the UFH controls should show a positive response (increased median P-selectin fluorescence) at 0.1 and 0.5 U/ml UFH but not at 100 U/ml UFH. The test was positive for cross-reactivity of the HIT antibodies with the 2-O desulfated heparin if an increase in platelet P-selectin expression occurred.

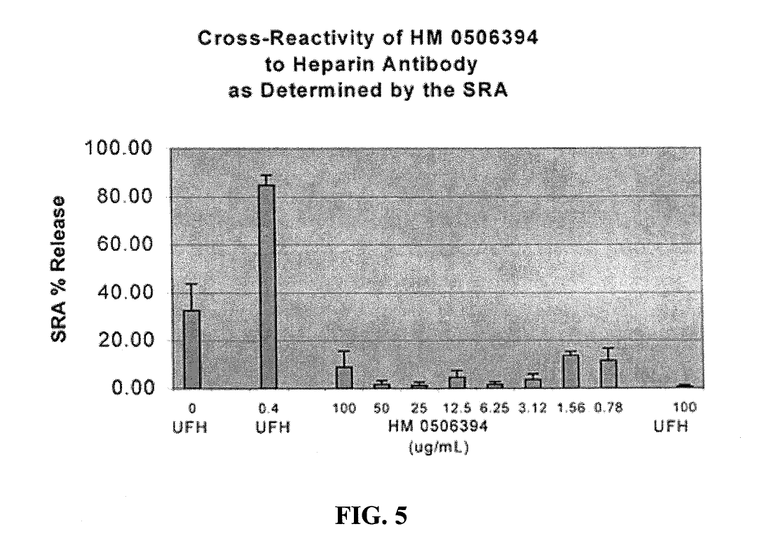

FIG. 5 shows that unfractionated heparin at the usual therapeutic anticoagulant concentration of 0.4 μg/ml elicited release of >80% of total radio labeled serotonin in this system. In contrast, the 2-O desulfated heparin (ODSH), studied in a range of concentrations from 0.78 to 100 μg/ml, failed to elicit substantial ¹⁴C serotonin release, indicating that this 2-O desulfated heparin does not interact with a pre-formed HIT antibody causing platelet activation. The interaction of regular heparin with the HIT antibody caused platelet activation. When ODSH was added with heparin to the HIT antibody, the ODSH prevented heparin from causing platelet activation.

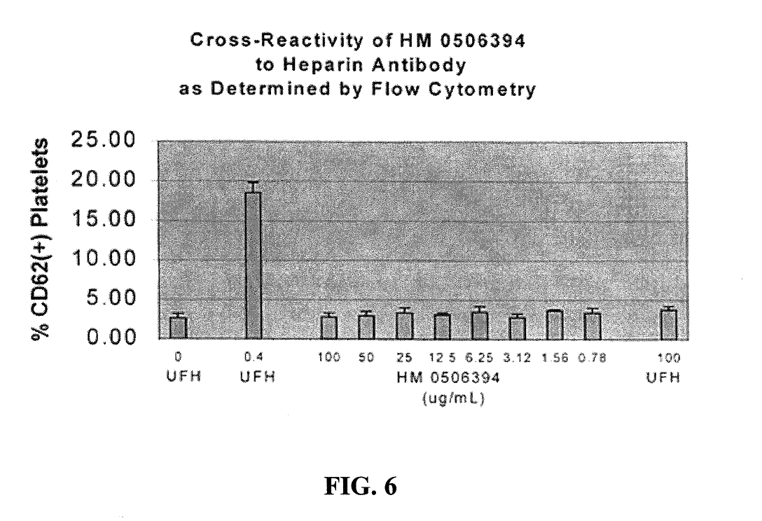

FIG. 6 shows that when unfractionated heparin at the usual therapeutic anticoagulant concentration of 0.4 μg/ml was incubated with platelets and HIT-antibody positive serum, there was prominent CD62 expression on the surface of approximately 20% of the platelets. Saline control incubations were characterized by low expression of CD62 (<2% of platelets). In contrast, 2-O desulfated heparin, studied at 0.78 to 100 μg/ml, did not increase CD62 expression levels above that observed in the saline control incubations. Furthermore, while 0.4 μg/ml unfractionated heparin produced substantial platelet microparticle formation, 2-O desulfated heparin at 0.78 to 100 μg/ml stimulated no level of platelet microparticle formation above that of the saline control incubations (<5% activity).

With a molecular weight of 11.8 kD and a degree of sulfation of about 1.0, ODS heparin would be predicted to elicit a HIT-like platelet activation response in the serotonin release and platelet microparticle formation assays. Thus, it is surprising and not predictable or obvious from the prior art that 2-O desulfated heparin does not react with HIT antibody and PF4 to activate platelets, and should not produce the HIT syndrome. This indicates that 2-O desulfated heparin is a safer therapeutic heparin analog for administration to patients for treatment of inflammatory and other conditions in need of heparin or heparin analog therapy, since 2-O desulfated heparin should not produce the serious and life-threatening HIT-2 syndrome.

More surprisingly, 2-O desulfated heparin actually suppresses platelet activation induced by HIT antibody and unfractionated heparin. For these amelioration experiments, the 2-O desulfated heparin employed was manufactured by the commercial process detailed in Example 3. The SRA and flow cytometry techniques, slightly modified from what was described above, were used to demonstrate this unique effect of the 2-O desulfated heparin.

SRA platelet-rich plasma was collected, prepared and labeled as previously described. The test system mixture incorporated both 5 μl of 2-O desulfated heparin (0, 0.78, 1.56, 3.13, 6.25, 12.5, 25, 50 and 100 μg/ml final concentrations) and 5 μl of unfractionated heparin (either 0.1 or 0.5 U/ml final concentrations). The SRA was positive for amelioration of the unfractionated heparin induced platelet activation by the 2-O desulfated heparin, if the UFH response was inhibited in the presence of 2-O desulfated heparin. Serotonin release <20% in the presence of UFH and 2-O desulfated heparin is considered complete amelioration.

For the flow cytometric analyses, whole blood was collected and prepared as previously described. The test system mixture incorporated both 25 μl of 2-O desulfated heparin (0, 0.78, 1.56, 3.13, 6.25, 12.5, 25, 50 and 100 μg/ml final concentrations) and 25 μl of unfractionated heparin (either 0.1 or 0.5 U/ml final concentrations). Heparin without 2-O desulfated heparin was used as the control (0, 0.1, 0.5 and 100 U/ml UFH final concentrations). Any test agent, such as 2-O desulfated heparin, is considered positive for amelioration if the 0.1 and 0.5 U/ml UFH response is inhibited. Complete amelioration occurred if the platelet activation response was equivalent to that of the 100 U/ml UFH control (no test agent, such as 2-O desulfated heparin, present).

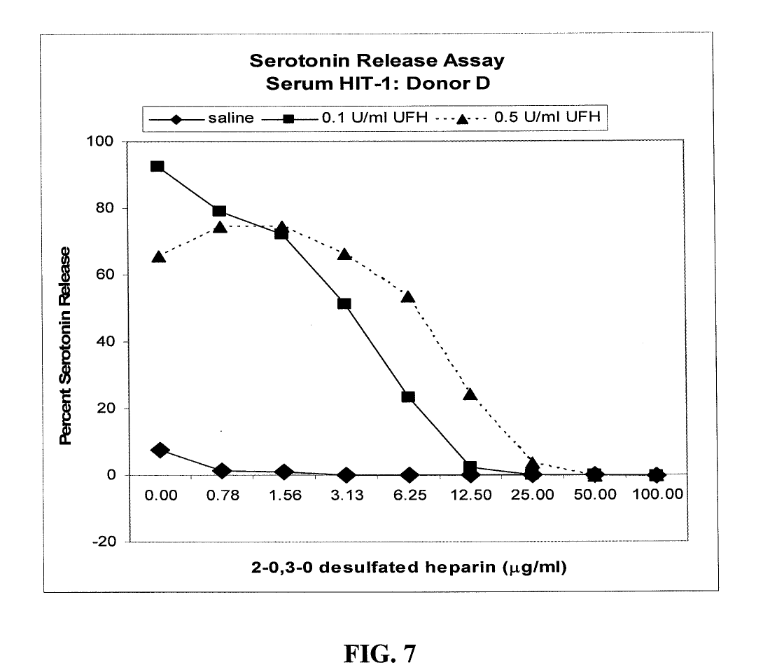

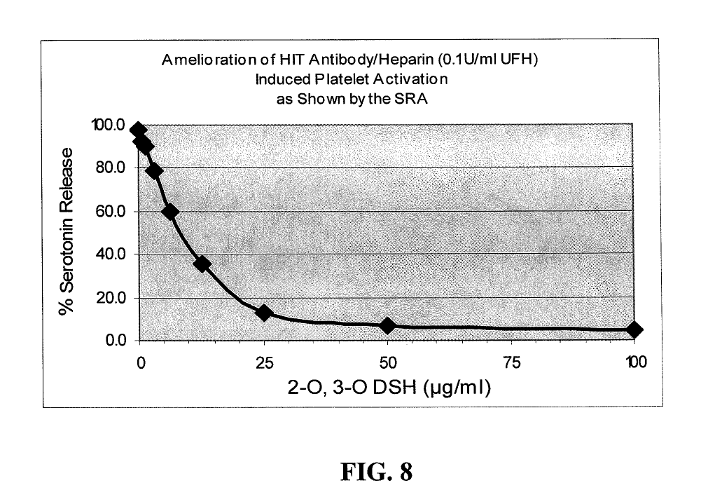

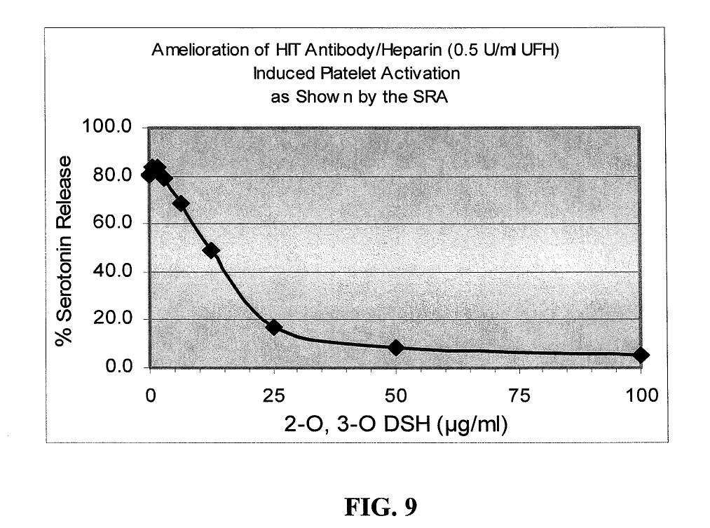

In the SRA, amelioration could be observed at concentrations of 2-O desulfated heparin, which is also 3-O desulfated, as low as 3.13 μg/ml. A higher concentration of the 2-O desulfated heparin (on average 6.25 μg/ml vs. 3.13 μg/ml) was needed to initiate amelioration in the 0.5 U/ml UFH system, compared to that needed in the 0.1 U/ml UFH system. Complete blockade of the HIT antibody/unfractionated heparin induced platelet activation was always obtained, but the concentrations of the 2-O desulfated heparin differed depending on the strength of the HIT antibody. FIG. 7 shows the results of amelioration of SRA using serum from a typical HIT patient. In most patient sera, complete amelioration (defined as <20% serotonin release) was observed at 12.5 μg/ml and higher concentrations of 2-O desulfated heparin. Composite graphs of the data obtained in studying SRA inhibition with sera from four different HIT patients is shown using the 0.1 U/ml UFH system (FIG. 8) and the 0.5 U/ml UFH system (FIG. 9). It can be seen that amelioration was initiated at 6.25 μg/ml and complete amelioration of the SRA response was achieved with 25 μg/ml of 2-O desulfated heparin. No platelet activation was observed in the presence of 50 μg/ml of 2-O desulfated heparin. Due to the consistency of the data, the error bars (standard error of the mean; SEM) do not show.

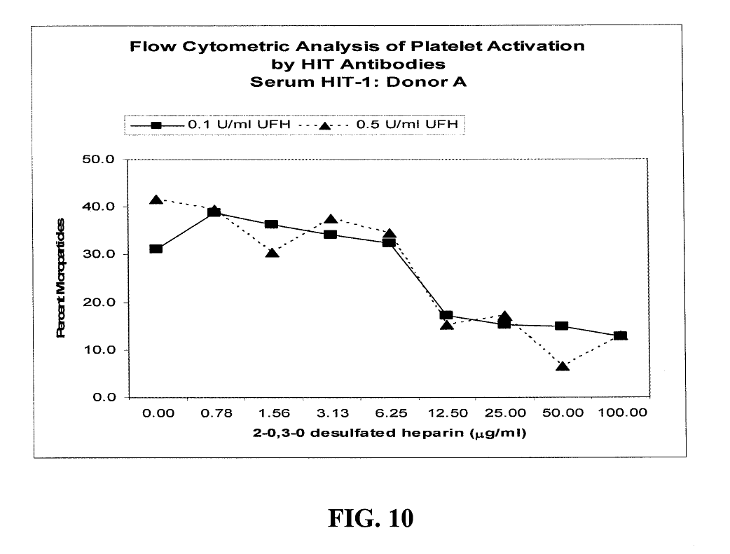

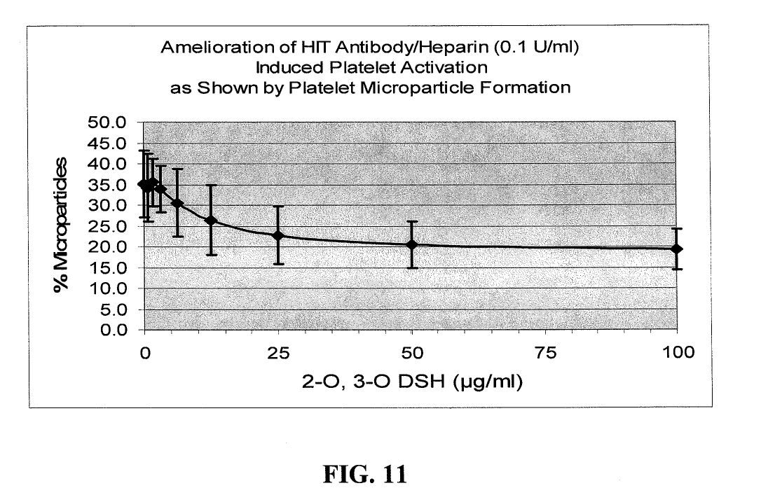

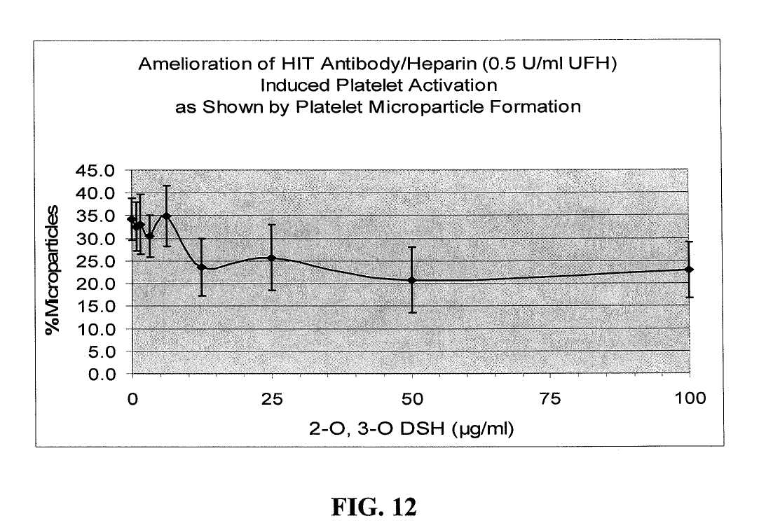

Evaluation of 2-O desulfated heparin for amelioration of platelet activation induced by HIT antibodies/unfractionated heparin using the flow cytometric analysis of platelet microparticle formation and cell surface P-selectin expression as a measure of platelet activation showed an amelioration effect in all test systems (defined as inhibition of the response obtained with 0.1 and 0.5 U/ml UFH response when no 2-O desulfated heparin was present). For platelet microparticle formation, amelioration was observed at concentrations of 2-O desulfated heparin as low as 6.25 μg/ml. There was no remarkable difference between the amelioration response observed in the 0.1 U/ml and the 0.5 U/ml UFH systems. On average, amelioration was initiated at 6.25 μg/ml 2-O desulfated heparin. Complete blockade of the platelet activation was always obtained, but the concentrations of 2-O desulfated heparin differed depending on the strength of the HIT antibody. FIG. 10 shows results of amelioration of HIT/unfractionated heparin induced platelet microparticle formation using serum from a typical HIT patient. Composite graphs of the data obtained in studying inhibition of platelet microparticle formation with sera from four different HIT patients is shown using the 0.1 U/ml UFH system (FIG. 11) and the 0.5 U/ml UFH system (FIG. 12). Complete amelioration (defined as platelet activation response equivalent to that of the 100 U/ml UFH control when the test agent 2-O desulfated heparin was not present) was observed from 6.25 μg/ml and higher concentrations of 2-O desulfated heparin. Over average, a concentration of 50 μg/ml 2-O desulfated heparin was needed to achieve complete remission of platelet microparticle formation.

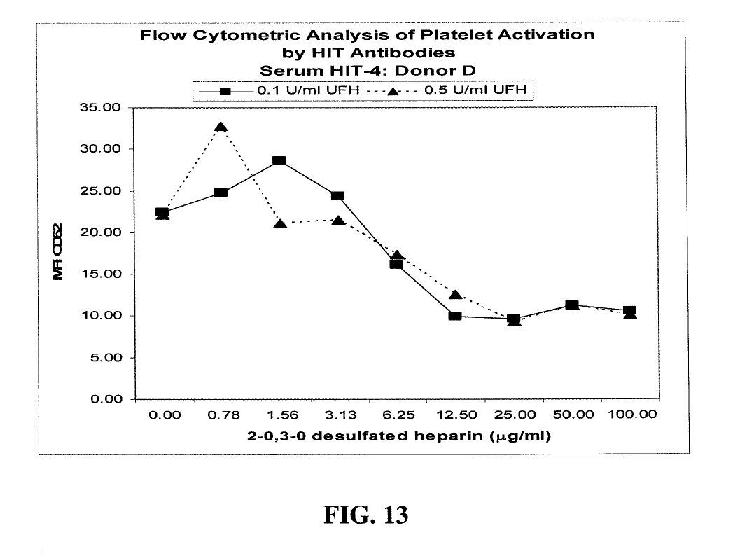

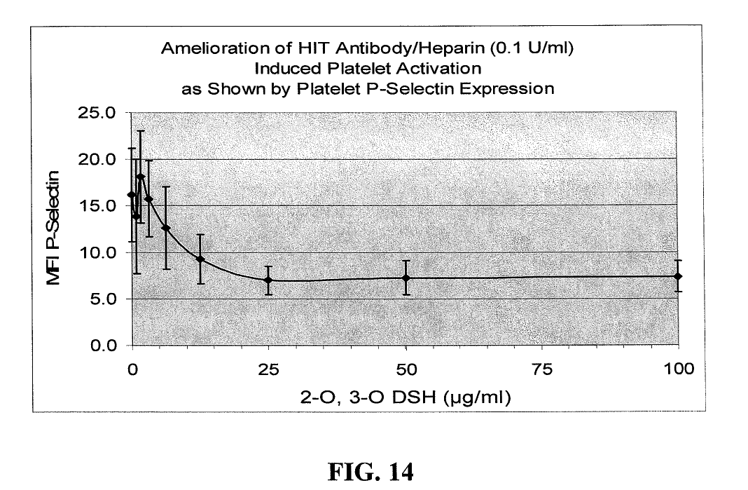

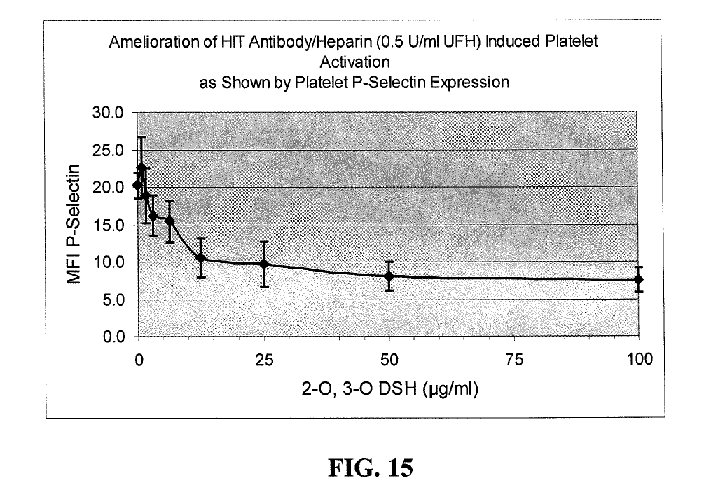

For P-selectin (CD62) expression, amelioration could be observed at concentrations of the 2-O desulfated heparin as low as 1.56 μg/ml. There was no remarkable difference between the amelioration response observed in the 0.1 U/ml and the 0.5 U/ml UFH systems. On average amelioration was initiated at 6.25 μg/ml 2-O desulfated heparin. Complete blockade of the platelet activation was always obtained, but the concentration of the 2-O desulfated heparin differed depending on the strength of the HIT antibody. FIG. 13 shows results of amelioration of HIT/unfractionated heparin induced platelet CD62 expression using serum from a typical HIT patient. Complete amelioration was observed from 6.25 μg/ml and higher concentrations of 2-O desulfated heparin. On average, a concentration of >25 μg/ml 2-O desulfated heparin was needed to achieve complete amelioration or suppression of platelet activation. Composite graphs of the data obtained in studying inhibition of platelet CD62 expression with sera from four different HIT patients is shown using the 0.1 U/ml UFH system (FIG. 14) and the 0.5 U/ml UFH system (FIG. 15). Amelioration was initiated at 6.25 μg/ml and complete amelioration of the platelet activation responses, measured by CD62 expression, was achieved with 50 μg/ml of 2-O desulfated heparin.

### Example 2

### Intravenous Injection of 2-O Desulfated Heparin to Achieve RAGE-Ligand Inhibiting Concentrations in the Bloodstream

To determine if levels of 2-O desulfated heparin reached sufficient concentration in vivo to suppress RAGE-ligand interactions and signaling, three groups of beagle dogs (n=4 each) were injected with 2-O desulfated heparin (ODSH) produced as in Example 3. Injections were given over 2 minutes in doses of 0 (saline control, group 1), 4 (group 2), 12 (group 3) and 24 mg/kg (group 4). Injections were performed 4 times daily for 10 days. On a daily basis, the total ODSH doses administered were 0, 16, 48 and 96 mg/kg. Whole blood was collected on study days 1, 2, 4, 6, and 8, at 15 minutes and 6 hours after the first injection of the day. Also, following the final ODSH injection, samples were collected at 15 minutes, and 1, 2, 4, 6 and 8 hours. All samples were collected in vacutainer tubes containing sodium citrate as an anticoagulant.

The concentration of ODSH was measured by a potentiometric assay developed for measurement of sulfated polysaccharides in biological fluids (see Ramamurthy N, et al., Anal Biochem 1999; 266:116-124). Cylindrical polycation sensitive electrodes were prepared as described previously (see Ramamurthy N, et al., Clin Chem 1998; 44:606-661). A cocktail with a composition of 1% (w/w) dinoylnaphthalene sulfonate, 49.5% (w/w) nitrophenyloctyl ether, and 49.5% (w/w) polyurethane M48 was prepared by dissolving components in distilled (THF) tetrahydrofuran (200 mg/ml). The resulting solution was dip coated onto the rounded ends of sealed glass capillary tubes protruding slightly from 1 inch pieces of Tygon tubing (i.d.=1.3-1.5 mm). After dip coating the solution 12 times at 15 minute intervals, the sensor bodies were dried overnight in a fume hood. On the day of use, the sensor bodies were soaked for at least one hour in PBS (Phosphate Buffered Saline) and the glass capillaries were carefully removed. The sensor body was then filled with PBS and a Ag/AgCl wire was inserted to complete the sensor. Sensors were used once and then discarded. Two sensors and a Ag/AgCl reference wire were connected to a VF-4 amplifier module (World Precision Instruments) that was interfaced to an NB-MIO analog/digital input/output board (National Instruments) in a Mac IIcx computer. The data was sampled at a 3 second interval and recorded with LabView 2.0 software. A titrant solution of 1 mg/ml protamine sulfate (clupeine form, Sigma) in PBS was prepared, and the titrant was delivered continuously via a syringe pump (Bioanalytical Systems). Titration end-points were computed using the Kolthoff method (See Sergeant EP, Chemical Analysis, Kolthoff I M, Elwing P J, eds. 69:362-364, 1985), followed by application of a subtractive correction factor equivalent to the protamine concentration required to reach the end point of the calibration curve.

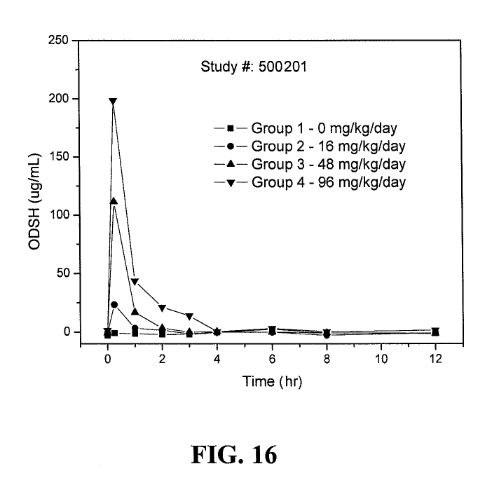

FIG. 16 shows concentrations of ODSH in plasma at timed collection intervals for the three dose groups and control. The average concentrations at various time points are shown in Table 1:

TABLE 1

| | ODS Heparin concentration (μg/ml) | | | |
|---|---|---|---|---|
| Sample | 0 mg/kg/day | 16 mg/kg/day | 48 mg/kg/day | 160 mg/kg/day |
| 15 min post injection | −0.1 ± 0.4 | 14.0 ± 0.9 | 50.4 ± 18.9 | 237.9 ± 26.5 |
| 1 hr post injection | 2.3 ± 0.7 | 2.4 ± 0.7 | 14.6 ± 0.9 | 86.4 ± 12.1 |
| 3 hr post injection | 0.9 ± 0.7 | 0.6 ± 0.7 | 1.7 ± 0.7 | 17.2 ± 0.8 |
| 4 hr post injection | 1.0 ± 0.7 | 0.4 ± 0.7 | −0.1 ± 0.7 | 10.7 ± 0.8 |
| 6 hr post injection | 1.8 ± 0.7 | 0.4 ± 0.7 | 1.4 ± 0.7 | 5.7 ± 0.8 |
| 8 hr post injection | 0.9 ± 0.7 | 0.1 ± 0.7 | 0.9 ± 0.7 | 2.1 ± 0.8 |
| 12 hr post injection | 1.7 ± 0.7 | 2.3 ± 0.7 | 0.9 ± 0.7 | 3.7 ± 0.8 |

Compartmental modeling was performed using WinNonlin version 4.1. Tables 2 and 3 display the pharmacokinetic parameters AUC (area under the curve), K10-HL (terminal half life), Cₘₐₓ(maximum concentration), CL (clearance), AUMC (area under the first moment curve), MRT (mean residence time), and Vₛₛ (volume of distribution at steady state) for each group respectively.

TABLE 2

| Dose | AUC | Vₛₛ | CL | Cₘₐₓ | Half-life |
|---|---|---|---|---|---|
| (mg/kg/day) | (hr*ug/mL) | (mL/kg) | (mL/hr/kg) | (ug/mL) | (hr) |
| 16 | 12.39 ± 1.92 | 127.23 ± 11.63 | 322.80 ± 49.98 | 23.28 ± 1.41 | 0.27 ± 0.06 |
| 48 | 59.90 ± 1.41 | 80.01 ± 1.11 | 200.35 ± 4.71 | 111.47 ± 1.03 | 0.28 ± 0.01 |
| 96 | 134.14 ± 10.96 | 97.39 ± 4.68 | 178.91 ± 14.63 | 197.60 ± 7.43 | 0.38 ± 0.04 |

TABLE 3

| Dose | Parameter | Units | Estimate | StdError | CV % |
|---|---|---|---|---|---|
| 16 | AUC | hr*ug/mL | 12.39 | 1.91 | 15.47 |
| 16 | K10-HL | hr | 0.27 | 0.05 | 21.17 |
| 16 | Cmax | ug/mL | 23.28 | 1.40 | 6.04 |
| 16 | CL | mL/hr/kg | 322.80 | 49.97 | 15.48 |
| 16 | AUMC | hr*hr*ug/mL | 6.43 | 1.98 | 30.91 |
| 16 | MRT | hr | 0.39 | 0.08 | 21.17 |
| 16 | Vss | mL/kg | 127.23 | 11.62 | 9.14 |
| 48 | AUC | hr*ug/mL | 59.89 | 1.40 | 2.35 |
| 48 | K10-HL | hr | 0.28 | 0.00 | 3.20 |
| 48 | Cmax | ug/mL | 111.47 | 1.03 | 0.92 |
| 48 | CL | mL/hr/kg | 200.35 | 4.70 | 2.35 |
| 48 | AUMC | hr*hr*ug/mL | 31.41 | 1.47 | 4.69 |
| 48 | MRT | hr | 0.39 | 0.01 | 3.20 |
| 48 | Vss | mL/kg | 80.01 | 1.10 | 1.38 |
| 96 | AUC | hr*ug/mL | 134.14 | 10.95 | 8.17 |
| 96 | K10-HL | hr | 0.38 | 0.03 | 10.44 |
| 96 | Cmax | ug/mL | 197.59 | 7.43 | 3.76 |
| 96 | CL | mL/hr/kg | 178.91 | 14.63 | 8.18 |
| 96 | AUMC | hr*hr*ug/mL | 89.79 | 14.54 | 16.20 |
| 96 | MRT | hr | 0.54 | 0.056 | 10.44 |
| 96 | Vss | mL/kg | 97.39 | 4.68 | 4.81 |

Levels of 2-O desulfated heparin were achieved that inhibit RAGE-ligand interactions and signaling and ameliorate all aspects of HIT platelet activation at injection doses of 4 mg/kg (16 mg/kg/day) and greater. With a load and infusion rate of approximately one-fifth the loading dose every hour, steady state levels are likely to be achievable in all cases.

### Example 3

### Production of 2-O Desulfated Heparin that is Nonanticoagulant and is Inhibitory for Human Leukocyte Elastase

USP porcine intestinal heparin was purchased from a commercial vendor [Scientific Protein Laboratories (SPL), Wanaukee, Wis.]. It was dissolved at room temperature (20±5° C.) to make a 5% (weight/volume) solution in deionized water. As a reducing step, 1% (weight/volume) sodium borohydride was added and agitated for 2 hours. The solution was then allowed to stand at room temperature for 15 hours. The pH of the solution was then alkalinized to greater than 13 by addition of 50% sodium hydroxide. The alkalinized solution was agitated for 2-3 hours. This alkalinized solution was then loaded onto the trays of a commercial lyophilizer and frozen by cooling to −40° C. A vacuum was applied to the lyophilizer and the frozen solution was lyophilized to dryness. The lyophilized product was dissolved in cold (<10° C.) water to achieve a 5% solution. The pH was adjusted to about 6.0 by slow addition of hydrochloric acid, with stirring, taking care to maintain the solution temperature at <15° C. The solution was then dialyzed with at least 10 volumes of water or subjected to ultrafiltration to remove excess salts and reducing agent. To the dialyzed solution, an amount of 2% sodium chloride (weight/volume) was added. The 2-O desulfated heparin product was then precipitated using one volume of hysol (denatured ethanol).

After the precipitation had settled for about 16 hours, the supernatant was siphoned off. The precipitate was re-dissolved in water to a 10% (weight/volume) solution. The pH was adjusted to 5-6 using hydrochloric acid or sodium hydroxide, the solution was filtered through a 0.2 μm filter capsule into a clean container. The filtered solution was then lyophilized to dryness. The resulting product made by this method had yields up to 1.5 kg.

The final product was a 2-O desulfated heparin with a pH of 6.4, a USP anticoagulant activity of about 6 U/mg and an anti-Xa anticoagulant activity of 1.9 U/mg. The product was free of microbial and endotoxin contamination and the boron content, measured by ICP-AES, was <5 ppm. This 2-O desulfated heparin thus produced has been tested in rats and dogs at doses as high as 160 mg/kg (of animal weight) daily for up to 10 days, with no substantial toxicity.

The resulting 2-O desulfated heparin was useful for inhibiting the enzymatic activity of human leukocyte elastase. This was tested by methods detailed in U.S. Pat. No. 5,668,188; U.S. Pat. No. 5,912,237; and U.S. Pat. No. 6,489,311. The inhibition of human leukocyte elastase (HLE) was measured by incubating a constant amount of HLE (100 pmol) with a equimolar amount of 2-O desulfated heparin (I/E ratio 1:1) for 30 minutes at 25° C. in 500 μL of Hepes buffer (0.125 M, 0.125% Triton X-100, pH 7.5) diluted to the final volume of 900 μL. The remaining enzyme activity was measured by adding 100 μL of 3 mM N-Suc-Ala-Ala-Val-nitroanalide (Sigma Chemical, St. Louis, Mo., made in dimethylsulfoxide). The rate of change in absorbance of the proteolytically released chromogen 4-nitroanline was monitored at 405 nm (nanometers). The percentage inhibition was calculated based upon enzyme activity without inhibitor. The 2-O desulfated heparin produced by above methods inhibited HLE >90% at a 1:1 enzyme to inhibitor molar ratio.

The bulk product was formulated into convenient unit dose vials of 50 mg/ml. This was accomplished by adding 2-O desulfated heparin to USP sterile water for injection, to make a 6.5% (weight/weight) solution. Sodium chloride and sterile water for injection were added to adjust the final osmolality to 280-300 mOsm, and the pH was adjusted to 7.1-7.3 using 1 N hydrochloric acid or sodium hydroxide, as needed. The solution was filtered and transferred to a sterile fill Class 100 area where unit dose glass vials were filled with 21 ml solution each, sealed, crimped and labeled.

### Example 4

### Reduction in Binding of Human U937 Monocytes to Immobilized RAGE by 2-O Desulfated Heparin and Other Sulfated Polysaccharides

The binding of the human monocyte cell line U937 to immobilized RAGE was used the study effect of heparin, low molecular weight heparan sulfate and modifications of heparin with low anticoagulant activity on interaction of RAGE with its ligands. U937 cells utilize the Mac-1 (CD11b/CD18) integrin as a counterligand to RAGE (Chavakis T, ibid.). Disruption of U937 cells to immobilized human RAGE can therefore serve as a model for specific RAGE-ligand interaction.

High-bind 96-well micro-titer plates were coated with 8 μg/ml protein A in 0.2 M carbonate-bicarbonate buffer, pH 9.4 (100 μl/well). Plates were washed with PBS containing 1% Bovine Serum Albumin (PBS-BSA). Each well was then coated with 50 μl of PBS containing a chimera (20 μg/ml) comprised of human RAGE conjugated to the Fc immunoglobulin chain (R&D Systems, Minneapolis, Minn.), and plates were incubated overnight at 4° C. to allow RAGE-Fc to adhere. Chimeras structured in such a fashion orient so that Fc is bound to the plate with RAGE oriented superior-most into the buffer within each well.

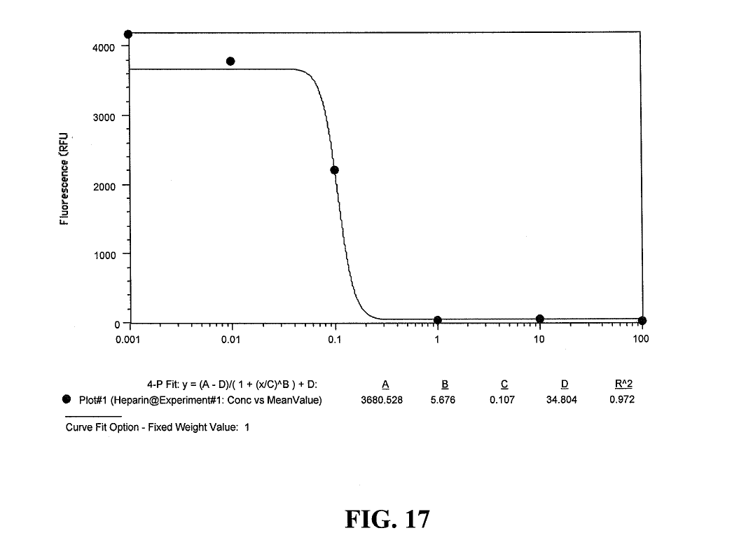

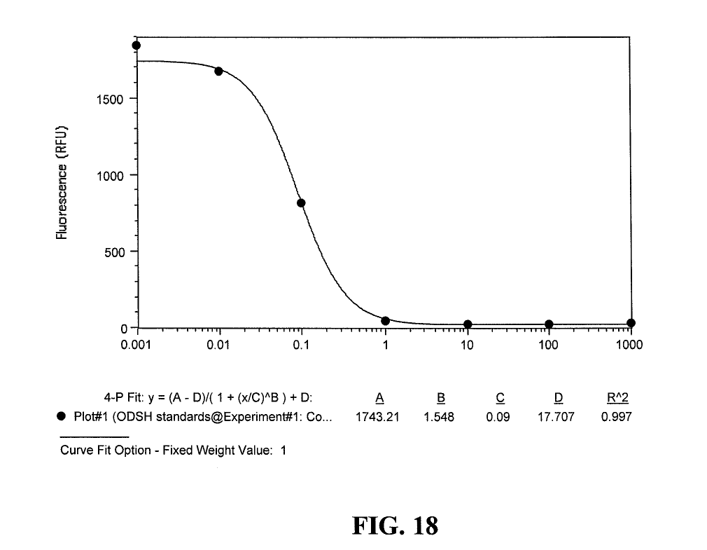

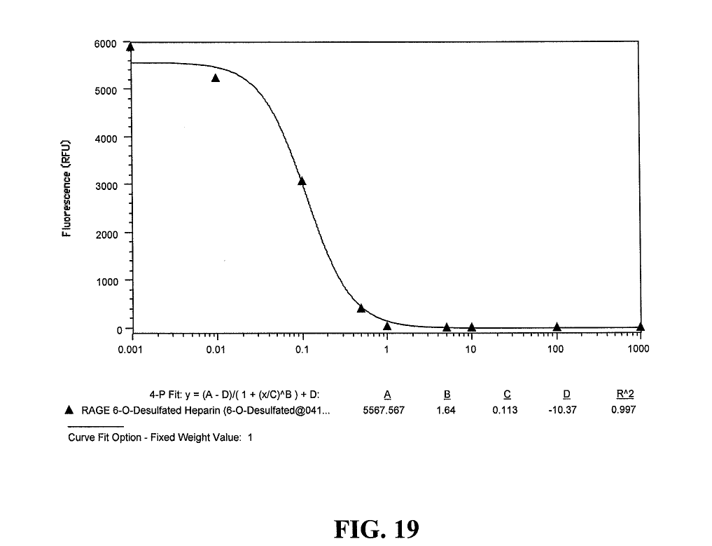

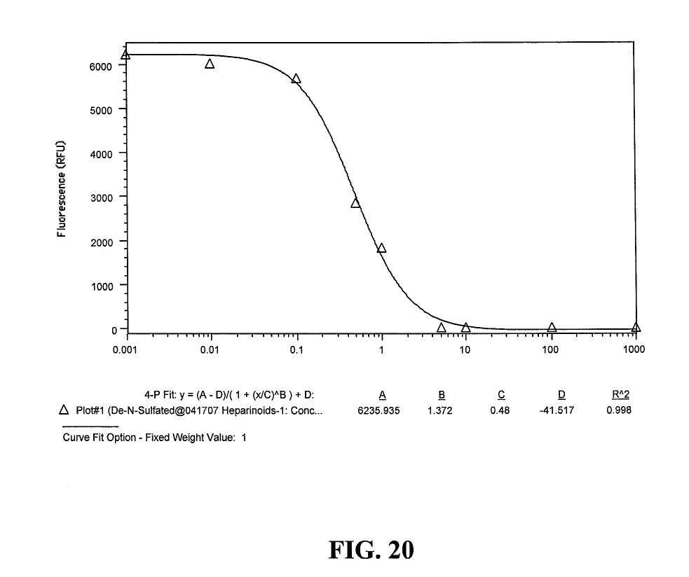

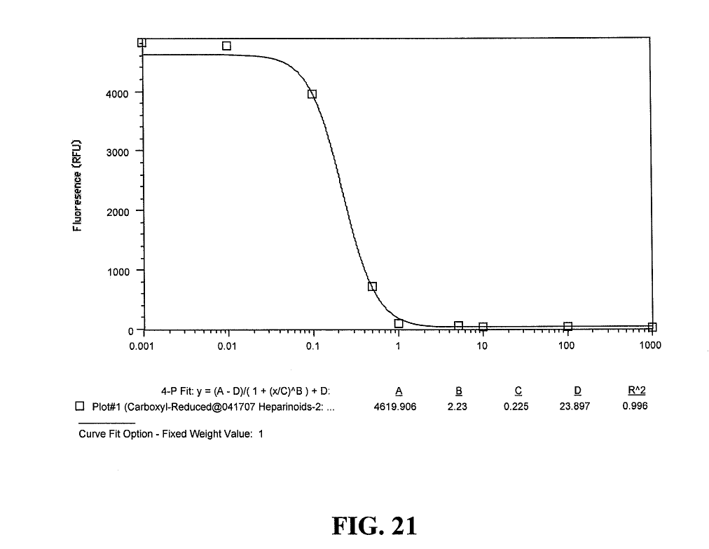

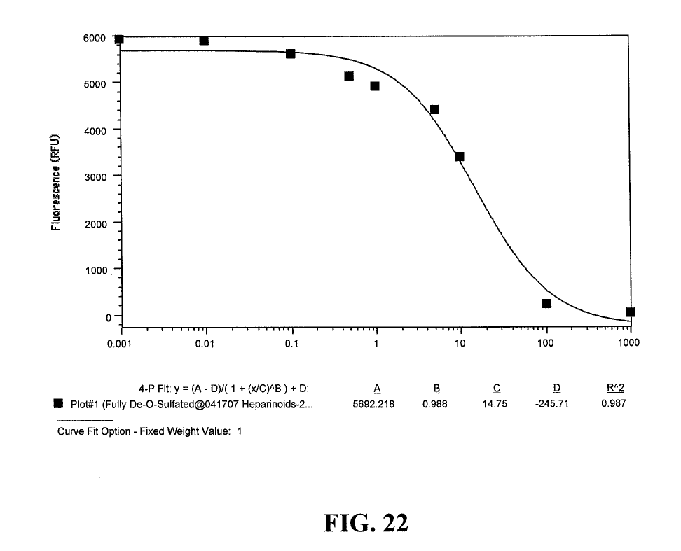

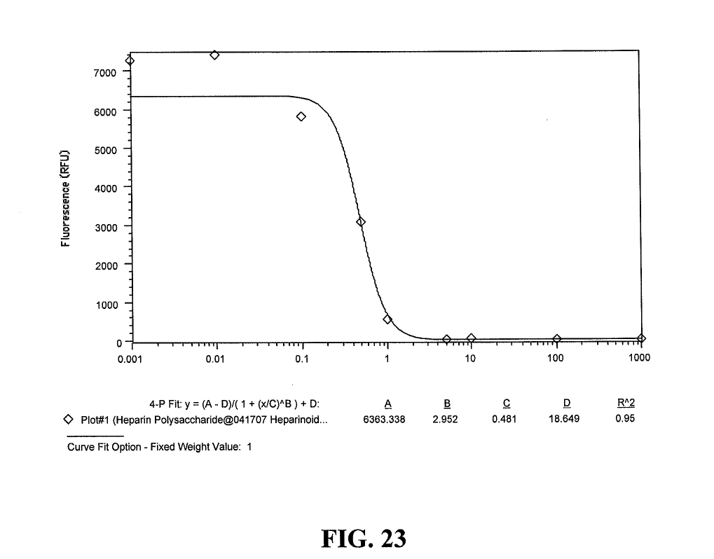

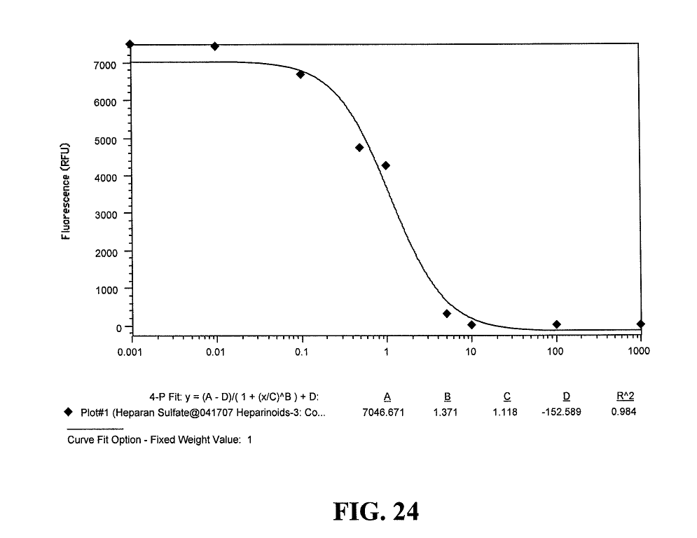

Following incubation, wells were washed twice with PBS-BSA, and 50 μl of PBS-BSA containing calcium, magnesium and serial dilutions of heparins, heparan sulfate or modified heparins (0-1000 μg/ml) was added to respective wells. To a select set of wells, 50 μl of 10 mM EDTA was added as a negative control. Wells were incubated at room temperature for 15 minutes. Thereafter, 50 μl of calcein-labeled U937 cells (10⁵ cells/well) were added to wells containing heparins, heparan sulfate, or modified heparins, and wells were incubated another 30 minutes at room temperature. Wells were then washed three times with PBS. Bound cells were lysed with Tris-TritonX-100 buffer, and fluorescence of each well was measured using excitation of 494 nm and emission of 517 nm. Fluorescence in relative units (RFU) was plotted against concentrations of glycosaminoglycans on a semi-logarithmic scale. Results are shown in FIG. 17 through FIG. 24. The 50% inhibitory concentration (IC₅₀) of each glycosaminoglycans against RAGE-ligand binding is shown in Table 4 below.

TABLE 4

| Type of Glycosaminoglycan | IC₅₀ (μg/ml) |
|---|---|
| Unfractionated porcine intestinal heparin | 0.107 |
| 2-O, 3-O desulfated heparin (ODSH) | 0.09 |
| 6-O desulfated heparin | 0.113 |
| N-desulfated heparin | 0.48 |
| Carboxyl-reduced heparin | 0.225 |
| Fully O-desulfated heparin | 14.75 |
| Low molecular weight heparin (MW 5,000 Da) | 0.481 |
| Heparan sulfate | 1.118 |

The most potent inhibitor of U937 cell binding to RAGE was 2-O desulfated heparin, which is also 3-O desulfated (ODSH). 2-O desulfated heparin inhibited RAGE-ligand interactions with an IC₅₀ concentration of only 0.09 μg/ml. 2-O desulfated heparin was much more potent (over 5 fold more potent) as an inhibitor of RAGE-ligand interaction than fully anticoagulant low molecular weight heparin (IC₅₀=0.481 μg/ml). 2-O desulfated heparin was an even more potent inhibitor of RAGE-ligand interaction than fully sulfated unfractionated heparin (IC₅₀=0.107 μg/ml). That 2-O desulfated heparin was more potent than even heparin was surprising in light of the fact that fully O-desulfated heparin (IC₅₀=14.75 μg/ml) demonstrated substantially reduced activity as an inhibitor of RAGE-ligand interactions. The use of 2-O desulfated heparin as an inhibitor of RAGE-ligand interactions would be clinically advantageous from the standpoint of safety. While unfractionated and low molecular weight heparins have full anticoagulant activity and can therefore be accompanied by adverse and unwanted risk of hemorrhage, 2-O desulfated heparin has low anticoagulant activity and carries substantially less risk of adverse hemorrhage when used as a clinical therapy. Unlike unfractionated heparin, other desulfated or carboxyl-reduced heparin derivatives, heparan sulfate or even low molecular weight heparins, 2-O desulfated heparin is also devoid of activity in producing heparin-induced thrombocytopenia, a rare but potentially lethal clinical complication of human treatment with glycosaminoglycans. Thus 2-O desulfated heparin and 2-O desulfated low molecular weight heparins and pentasaccharides offer superior safety and efficacy as clinical drug therapies for the inhibition of RAGE-ligand interactions and signaling.

### Example 5

### Reduction in Binding of AMJ2C-11 Alveolar Macrophages to Immobilized RAGE by 2-O Desulfated Heparin

The binding of the mouse alveolar macrophage cell line AMJ2C-11 to immobilized RAGE was used the study effect of 2-O desulfated heparin on interaction of RAGE with its ligands. AMJ2C-11 cells also utilize the Mac-1 (CD11b/CD18) integrin as a counterligand to RAGE. Disruption of AMJ2C-11 cells to immobilized human RAGE can also therefore serve as a model for specific RAGE-ligand interaction.

High-bind 96-well micro-titer plates were coated with 8 μg/ml protein A in 0.2 M carbonate-bicarbonate buffer, pH 9.4 (100 μl/well). Plates were washed with PBS containing 1% Bovine Serum Albumin (PBS-BSA). Each well was then coated with 50 μl of PBS containing a chimera (20 μg/ml) comprised of human RAGE conjugated to the Fc immunoglobulin chain (R&D Systems, Minneapolis, Minn.), and plates were incubated overnight at 4° C. to allow RAGE-Fc to adhere. Chimeras structured in such a fashion orient so that Fc is bound to the plate with RAGE oriented superior-most into the buffer within each well.

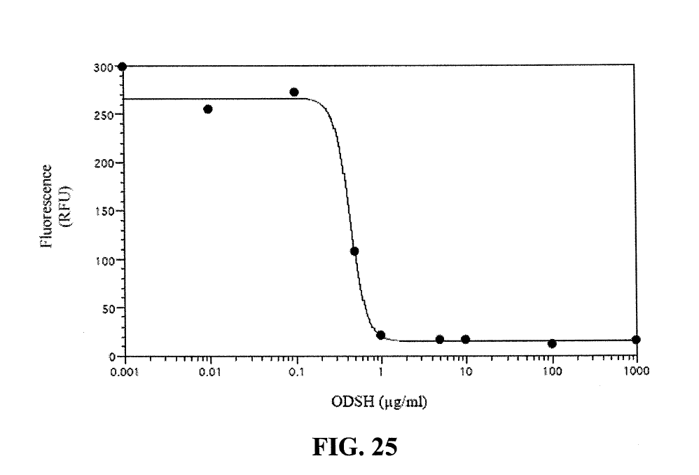

Following incubation, wells were washed twice with PBS-BSA, and 50 μl of PBS-BSA containing calcium, magnesium and serial dilutions of 2-O desulfated heparin (0-1000 μg/ml) was added to respective wells. To a select set of wells, 50 μl of 10 mM EDTA was added as a negative control. Wells were incubated at room temperature for 15 minutes. Thereafter, 50 μl of calcein-labeled AMJ2C-11 cells (10⁵ cells/well) were added to wells containing 2-O desulfated heparin, and wells were incubated another 30 minutes at room temperature. Wells were then washed three times with PBS. Bound cells were lysed with Tris-TritonX-100 buffer, and fluorescence of each well was measured using excitation of 494 nm and emission of 517 nm. Fluorescence in relative units (RFU) was plotted against concentrations of glycosaminoglycans on a semi-logarithmic scale. Results are shown in FIG. 25. The 50% inhibitory concentration (IC₅₀) of 2-O desulfated heparin against RAGE-ligand binding is shown in FIG. 25 to be 0.45 μg/ml.

The use of 2-O desulfated heparin as an inhibitor of RAGE-ligand interactions involving alveolar macrophages would be clinically advantageous from the standpoint of safety. While unfractionated and low molecular weight heparins have full anticoagulant activity and can therefore be accompanied by adverse and unwanted risk of hemorrhage, 2-O desulfated heparin has low anticoagulant activity and carries substantially less risk of adverse hemorrhage when used as a clinical therapy. Unlike unfractionated heparin, other desulfated or carboxyl-reduced heparin derivatives, heparan sulfate or even low molecular weight heparins, 2-O desulfated heparin is also devoid of activity in producing heparin-induced thrombocytopenia, a rare but potentially lethal clinical complication of human treatment with glycosaminoglycans. Thus 2-O desulfated heparin and 2-O desulfated low molecular weight heparins and pentasaccharides offer superior safety and efficacy as clinical drug therapies for the inhibition of RAGE-ligand interactions and signaling.

### Example 6

### Reduction in Binding of RAGE Ligands to Immobilized RAGE by 2-O Desulfated Heparin

Solid phase binding assays were used to study the ability of 2-O desulfated heparin to inhibit RAGE binding to its ligands. For studies of the effect of heparinoids on RAGE binding to its ligands, polyvinyl 96-well plates were coated with 5 μg/well of specific ligand (CML-BSA, HMGB-1 or S100b calgranulin). Plates were incubated overnight at 4° C. and washed thrice with PBS-0.05% Tween-20 (PBST). Separately, RAGE-Fc chimera (100 μL containing 0.5 μg/ml in PBST-0.1% BSA) was incubated with an equal volume of serially diluted ODSH (0.001 to 1,000 μg/ml in PBST-BSA) overnight at 4° C. The following day, 50 μL of RAGE-ODSH mix was transferred to each respective ligand-coated well and incubated at 37° C. for 2 h. Wells were then washed four times with PBST. To detect bound RAGE, 50 μL of anti-RAGE antibody (0.5 μg/ml) was added to each well, the mixture was incubated for 1 h at room temperature, and wells were washed again four times with PBST. Horse-radish peroxidase conjugated secondary antibody (50 μL per well) was added, wells were incubated for 1 h at room temperature, and then washed once with PBST. A colorimetric reaction was initiated by addition of 50 μL of TMB and terminated after 15 min by addition of 50 μL of 1 N HCl. Absorbance at 450 nm was read using an automated microplate reader.

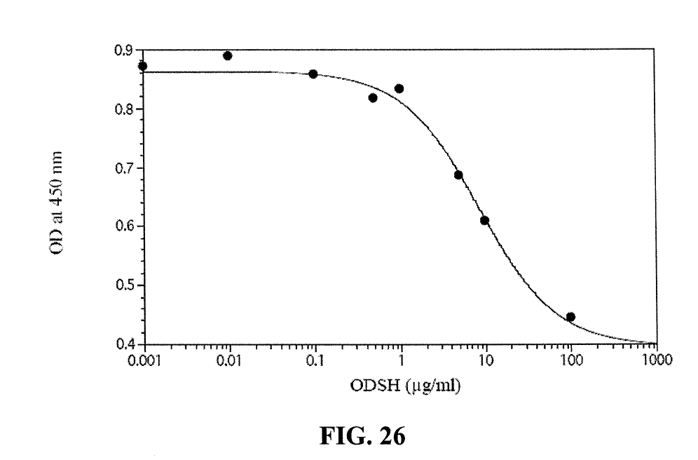

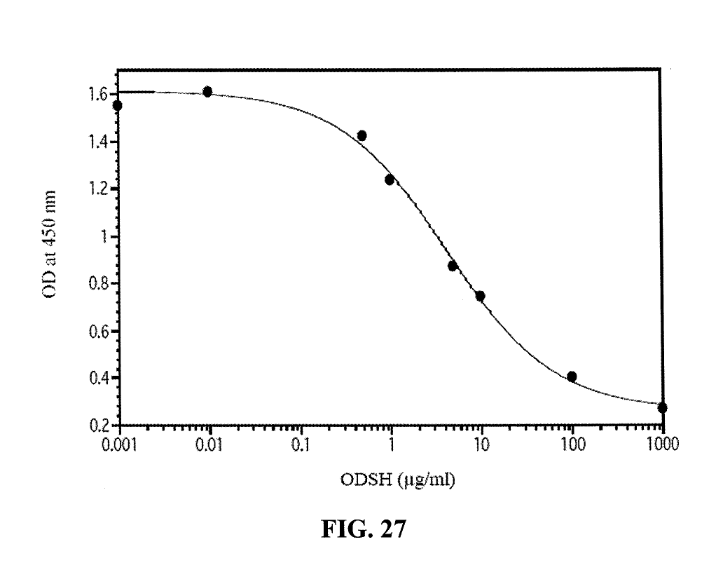

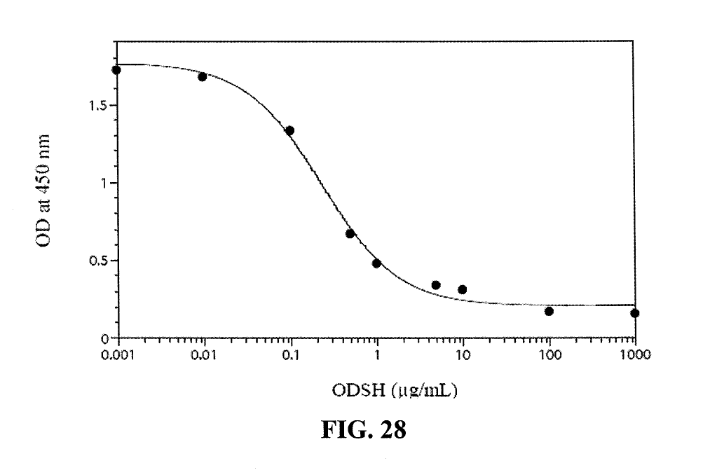

2-O desulfated heparin effectively inhibited RAGE interaction with the AGE product carboxymethyl-lysine-BSA (FIG. 26, IC₅₀=8.6 μg/ml), with S100b calgranulin (FIG. 27, IC₅₀=4.2 μg/ml) and with HMGB-1 or amphoterin (FIG. 28, IC₅₀=2.5 μg/ml), indicating that this nonanticoagulant heparin derivative blocks RAGE interaction with the full spectrum of ligands targeting this critically important pro-inflammatory receptor.

The use of 2-O desulfated heparin as an inhibitor of RAGE interactions with the ligands AGE products, S100 calgranulins or HMGB-1 would be clinically advantageous from the standpoint of safety. While unfractionated and low molecular weight heparins have full anticoagulant activity and can therefore be accompanied by adverse and unwanted risk of hemorrhage, 2-O desulfated heparin has low anticoagulant activity and carries substantially less risk of adverse hemorrhage when used as a clinical therapy. Unlike unfractionated heparin, other desulfated or carboxyl-reduced heparin derivatives, heparan sulfate or even low molecular weight heparins, 2-O desulfated heparin is also devoid of activity in producing heparin-induced thrombocytopenia, a rare but potentially lethal clinical complication of human treatment with glycosaminoglycans. Thus 2-O desulfated heparin and 2-O desulfated low molecular weight heparins and pentasaccharides offer superior safety and efficacy as clinical drug therapies for the inhibition of RAGE-ligand interactions and signaling.

Many modifications and other embodiments of the inventions set forth herein will come to mind to one skilled in the art to which these inventions pertain having the benefit of the teachings presented in the foregoing descriptions. Therefore, it is to be understood that the inventions are not to be limited to the specific embodiments disclosed and that modifications and other embodiments are intended to be included within the scope of the appended claims. Although specific terms are employed herein, they are used in a generic and descriptive sense only and not for purposes of limitation. Unless otherwise specified, all parts and percents are by weight and all temperatures are in Degrees Centigrade.

1. A method of inhibiting interaction or signaling of a ligand with the receptor for advanced glycation end products (RAGE) comprising contacting the receptor with 2-O desulfated heparin.

2. The method of claim 1, comprising contacting the receptor with 2-O, 3-O desulfated heparin.

3. The method of claim 1, wherein the ligand is selected from the group consisting of advanced glycation end-products (AGEs), Alzheimer's β peptide, Amyloid proteins, S100 calgranulins, HMGB-1 (amphoterin), and Mac-1 integrin.

4. The method of claim 1, comprising inhibiting interaction or signaling of an AGE with RAGE by contacting RAGE with 2-O desulfated heparin.

5. The method of claim 1, comprising inhibiting interaction or signaling of an S100 calgranulin with RAGE by contacting RAGE with 2-O desulfated heparin.

6. The method of claim 1, comprising inhibiting interaction or signaling of HMGB-1 with RAGE by contacting RAGE with 2-O desulfated heparin.

7. The method of claim 1, comprising inhibiting interaction or signaling of Mac-1 integrin with RAGE by contacting RAGE with 2-O desulfated heparin.

8. A method of treating a subject with a condition mediated by ligation of the receptor for advanced glycation end products (RAGE) comprising administering to the subject 2-O desulfated heparin in an amount effective to inhibit ligation of the receptor by a ligand.

9. The method of claim 8, comprising administering to the subject 2-O, 3-O desulfated heparin.

10. The method of claim 8, wherein the condition is selected from the group consisting of diabetes, inflammation, renal failure, aging, systemic amyloidosis, Alzheimer's disease, inflammatory arthritis, atherosclerosis, colitis, periodontal diseases, psoriasis, atopic dermatitis, rosacea, multiple sclerosis, chronic obstructive pulmonary disease (COPD), cystic fibrosis, photoaging of the skin, age-related macular degeneration, and acute lung injury.

11. The method of claim 8, wherein the ligand is selected from the group consisting of advanced glycation end-products (AGEs), Alzheimer's β peptide, Amyloid proteins, S100 calgranulins, HMGB-1 (amphoterin), and Mac-1 integrin.

12. The method of claim 8, wherein the condition is characterized by activation or expression of one or more enzymes or pathways selected from the group consisting of p21 ras, ERK 1/2 MAP kinases, JNK kinases, rho GTPases, phosphoinositol-3 kinase, JAK/STAT pathway, NF-κB, CREB, TNF-α, IL-1, IL-6, IL-8, GMCSF, iNOS, ICAM-1, E-selectin, VCAM-1, and VEGF.
